(19) United States
(12) Patent Application Publication
(10) Pub. No.: US 2019/0203270 A1
(43) Pub. Date: Jul. 4, 2019

(54) METHODS AND KITS FOR ANALYZING DNA BINDING MOIETIES ATTACHED TO DNA

(71) Applicant: Yeda Research and Development Co., Ltd., Rehovot (IL)

(72) Inventors: Ido AMIT, Rehovot (IL); David LARA-ASTIASO, Rehovot (IL); Meital GURY, Rehovot (IL)

(73) Assignee: Yeda Research and Development Co., Ltd., Rehovot (IL)

(21) Appl. No.: 16/319,499

(22) PCT Filed: Jul. 24, 2016
(86) PCT No.: PCT/IL2016/050808

§ 371 Date: Jan. 22, 2019

(51) Int. Cl. C12Q 1/6811; C12Q 1/6818; C12Q 1/6876

(52) U.S. Cl. CPC C12Q 1/6811; C12Q 1/6818; C12Q 2600/156; C12Q 2600/154; C12Q 1/6876

(57) ABSTRACT

Methods of analyzing DNA molecules in a cell sample are provided. The DNA molecules have DNA binding moiety signatures which are defined by at least two non-identical DNA binding moieties. Kits for analyzing the DNA are also provided.

### FIELD AND BACKGROUND OF THE INVENTION

The present invention, in some embodiments thereof, relates to methods and kits for analyzing DNA binding moieties attached to DNA, and, more particularly, but not exclusively for analyzing histone binding in a cell.

Understanding how the genome functions and is self-regulated beyond the evident DNA sequence is one of the major challenges of modern biology. The current view presents an epigenome partitioned into distinct functional elements, including promoters, enhancers, polycomb-repressed regions, gene bodies and insulators, which interact to produce stable expression patterns supporting defined cellular fates. Most of these elements are characterized by enrichment of particular post-translational modifications (PTMs) to the amino acids in the histone tails. For example, monomethylation of histone 3 lysine 4 (H3K4me1) is enriched in both poised and active enhancers, while the addition of acetylation on lysine 27 signifies active enhancers. This led to the hypothesis of a complex histone code in which combinations of different PTMs mark different functional genomic regions that are read, written and erased by chromatin modifiers to regulate cell fate. Combinations of histone modifications may also act in a cumulative manner or may exhibit more complex interactions.

In the last decade, genome-wide profiles of dozens of histone PTMs across different cell populations were analyzed using chromatin immunoprecipitation followed by massively parallel sequencing (ChIP-seq). Profiling of embryonic stem cells (ESC) identified a bivalent “poised state” marking important developmental genes characterized by an active (H3Kme3) and a repressive (H3K27me3) mark on the same loci. For some regions, these modifications have indeed been confirmed to co-occur on the same nucleosome using sequential ChIP. The bivalent chromatin state is crucial to understanding genome regulation during development, but current sequential ChIP methods are limited to measurements of only a few loci.

Additional background art includes Blecher Gonen et al., Nature Protocols, 8, 539-554 (2013), US Patent Application No. 20140024052, WO 2013/134261, WO 2002/014550, WO2012/047726 and WO 2015/159295.

### SUMMARY OF THE INVENTION

According to an aspect of some embodiments of the present invention there is provided a method of analyzing DNA molecules in a cell sample, the DNA molecules having DNA binding moiety signatures which are defined by at least two non-identical DNA binding moieties, the method comprising:

(a) labeling DNA molecules of the cell sample with a label that indexes the identity of at least two of the DNA binding moieties of the signatures so as to generate subpopulations of differentially labeled DNA molecules, each subpopulation being in a separate container, wherein the labeling indexes one DNA binding moiety per DNA molecule;

(b) labeling the differentially labeled molecules with another label which indexes the identity of another DNA binding moiety of the signature; and

(c) analyzing the DNA comprising the first label and the second label.

According to an aspect of some embodiments of the present invention there is provided a kit for immunoprecipitating a DNA-protein complex comprising:

(i) at least one antibody which specifically binds to a transcription factor;
(ii) at least one antibody which specifically binds to a post-translationally modified histone; and
(iii) a DNA labeling agent.

According to embodiments of the present invention, the label is a nucleic acid label.

According to embodiments of the present invention, the labeling of step (a) comprises attaching no more than one label per DNA molecule.

According to embodiments of the present invention, the labeling comprises end-labeling.

According to embodiments of the present invention, the labeling of step (b) comprises attaching no more than one label per DNA molecule.

According to embodiments of the present invention, the method further comprises repeating step (b) using an additional label prior to step (c).

According to embodiments of the present invention, neither the first DNA binding moiety nor the second DNA binding moiety bind to more than 50% of the DNA of the sample.

According to embodiments of the present invention, the method further comprises pooling the subpopulations to generate a pooled sample of differentially labeled DNA molecules following step (a) and prior to step (b).

According to embodiments of the present invention, the method further comprises shearing the DNA of the cell sample prior to step (a);

According to embodiments of the present invention, the analyzing comprises sequencing the DNA.

According to embodiments of the present invention, the method further comprises analyzing the DNA binding moieties following step (b).

According to embodiments of the present invention, the DNA is no longer than 500 bases.

According to embodiments of the present invention, the DNA binding moiety is a DNA binding protein.

According to embodiments of the present invention, the DNA binding protein is a histone.

According to embodiments of the present invention, the DNA binding protein is a transcription factor.

According to embodiments of the present invention, the DNA binding moiety is a drug.

According to embodiments of the present invention, the sample is derived from cells of a single type or line.

According to embodiments of the present invention, the histone is a post-translationally modified histone.

According to embodiments of the present invention, the post-translationally modified histone is a methylation or acetylation.

According to embodiments of the present invention, the post-translationally modified histone is selected from the group consisting of H3K4me1, H3K4me2, H3K4me3 and H3K27ac.

According to embodiments of the present invention, the kit further comprises an antibody for immobilizing at least 50% of the chromatin of a cell.

According to embodiments of the present invention, the kit further comprises at least one agent selected from the group consisting of an RNA polymerase, a DNAse and a reverse transcriptase.

According to embodiments of the present invention, the kit further comprises a plurality of barcode DNA sequences.

According to embodiments of the present invention, the kit further comprises a solid support for immobilizing the at least one antibody.

According to embodiments of the present invention, the kit further comprises at least one component selected from the group consisting of a crosslinker, a protease enzyme and a ligase.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

Some embodiments of the invention are herein described, by way of example only, with reference to the accompanying drawings and images. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-F illustrate the steps of combinatorial indexed chromatin immunoprecipitation (Co-ChIP). (A) Schematic diagram of the pipeline for combinatorial ChIP-seq (Co-ChIP) involving direct histone tagging and dual barcoding strategy for PTM combinations. (B) Normalized conventional ChIP-seq profiles for the Hoxa loci of H3K4me3 (green), H3K27ac (blue) and H3K27me3 (red), and the Co-ChIP profiles of active promoters (H3K4me3-H3K27ac), poised promoters (H3K4me3-H3K27me3) and H3K27ac-H3K27me3 as control. (C) Scatterplot comparing Co-ChIP reads counts of the pair H3K4me3^{1Ab}-H3K27ac^{2Ab} compared to switching the order of antibodies to H3K27ac^{1Ab}-H3K4me3^{2Ab} (D) Scatterplot comparing Co-ChIP read counts of H3K27ac with H3K4me3 using two different H3K4me3 antibodies (Millipore and Abcam). (E) Co-ChIP profiles with the primary antibody for 3 transcription factors, CTCF, Cebpb and Pu.1 together and the secondary antibody for 3 histone modifications (H3K4me3, H4K4me2 and H3K27ac). Shaded in blue are regions in which the co-occurrence of TF are specific for a subset of the histone marks. (F) Peak counts for TF-PTM pairs, including CTCF (blue), Cebpb (purple) and PU.1 (red) as primary antibodies and H3K27ac, H3K4me2 and H3K4me3 as secondary antibodies. Peak counts of conventional ChIP²⁴ on the TFs are indicated.

FIGS. 2A-B. Pairwise co-occurrence of 70 histone PTMs. (A) Heatmap of pairwise correlations between conventional ChIP profiles of 14×5 PTMs pairs. Negative correlations in blue and positive correlations in yellow (left). Heatmap showing relative abundance of each PTM pair (PTM¹-PTM²) normalized to H3 reference (PTM¹-H3) (right). (B) Heatmap showing K-means (k=15) clustering of normalized Co-ChIP reads in 158,200 2 Kb windows in the genome. Rows are clustered using hierarchical clustering, each row corresponds to a different PTM pair (37 pairs presented; Methods) as indicated on the left. Top panel displays region-by-region distance from the nearest transcription start site (TSS) using sliding window average smoothing. Lower panel show a bar-plot of 0.25 and 0.75 percentiles of mRNA-seq expression of the nearest gene (filled box) per cluster, whiskers extend to 0.05 and 0.95 percentile.

FIGS. 3A-E. Inclusive and exclusive interactions between histone marks. (A) Schematic model of inclusive, independent and exclusive interactions between modifications. Upper panel describes the possible configurations of histone marks co-occurrence and the transitions between states, lower panel shows the outcome of measurements as they would appear using conventional ChIP versus Co-ChIP. (B) Conventional ChIP profiles of 2 histone modifications, their Co-ChIP profile and the predicted co-occurrence profile using a multiplicative model, lower track shows the log ratio of Co-ChIP and prediction (positive=inclusion or negative=exclusion). Upper panel displays H4K12ac and H3K4me2, lower panel displays H3K27ac and H3K4me3 pair. Data was smoothed using a moving average filter with span=5 (C) Bar plots of TSS distances of 9 histone pairs divided by type of interaction (inclusion, independent, or exclusion). Histone acetylation (H3K27, H3K9 and H4K12) together with H3K4me1, me2 and me3. (D) Profiles of HDAC1 ChIP-seq, Co-ChIP of H3K4me2-H3K27ac, the predicted co-occurrence based on single ChIP and the log ratio of Co-ChIP to predictions. (E) Histogram of HDAC1 ChIP-seq read counts at genomic regions showing exclusive interactions between H3K4me2 and H3K27ac tend to have increased HDAC1 binding compared to inclusive interactions.

FIGS. 4A-B. Bivalent domains characterization of distinct ES cells states. (A) Co-ChIP profiles of bivalent domains, H3K4me3-H3K27me3 (red), and active domains, H3K4me3-H3K27ac (green) for ground state naïve mES cells (2i/LIF) and relatively primed mES cells (Serum/LIF) in several genomic regions. The genes are indicated on top of each panel and are marked as blue arrows. (B) Scatterplot of H3K4me3-H3K27me3 Co-ChIP read counts for bivalent regions of ES cells from Serum/LIF and 2i/LIF conditions in a 2 kb window centered around the TSS (top) and active promoters (H3K4me3-H3K27 ac) (bottom).

FIGS. 5A-C. Bivalency dynamics from ES cells to adult tissues. (A) Pairwise correlation of H3K4me3-H3K27me3 Co-ChIP log reads count in 23,167 enriched regions between ES and four adult tissues (top). Venn diagram of peak overlap between the bivalent regions in ES, brain and union of kidney, liver and ling data sets (bottom). (B) Heatmap showing 23,167 bivalent regions clustered with K-means (k=8) of log reads count of H3K4me3-H3K27me3 co-occurrence (Methods). Selected genes are shown on the right. (C) Profiles of H3K4me3-H3K27me3 levels in 7 gene loci: Brachyury bivalent in ES and lost in all tissues, Pax6 lost only in brain, Sox9 lost only in kidney, Hoxa7 lost only in lung. Nrg1, Rhoc and Six4 are examples of de novo acquired bivalency.

FIG. 6 is an example of a Y shaped adapter which is tagged at the 5′ end of the library after ligation.—Read 1 (green): reads the ChIPed DNA sequence (INSERT)

FIG. 7 is an example of a Y shaped adapter which is tagged at the 3′ end of the library after ligation—i7 (blue): reads the 8 mer barcode.

### DESCRIPTION OF SPECIFIC EMBODIMENTS OF THE INVENTION

The present invention, in some embodiments thereof, relates to methods and kits for analyzing DNA binding moieties attached to DNA, and, more particularly, but not exclusively for analyzing histone binding in a cell.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

Recent computational approaches have annotated the genome in higher resolution using probabilistic models to define regions sharing complex combinations of histone modifications or ‘chromatin states’. Despite these important findings, computational methods to extrapolate chromatin states assume that histone modifications measured from a bulk population of cells co-occupy the same DNA molecule and hence may over look cellular heterogeneity and dynamics. Mass spectrometry on the other hand, provides a quantitative and comparative approach for studying histone modifications and measuring their co-occurrence on the same peptide; yet these techniques do not provide information on the genomic location at which these interactions occur. Due to the technical limitations of both ChIP-seq and Mass-Spec technologies, it is not possible to empirically probe genome-wide interactions between different histone modifications on the same DNA molecule and identify relationships between them.

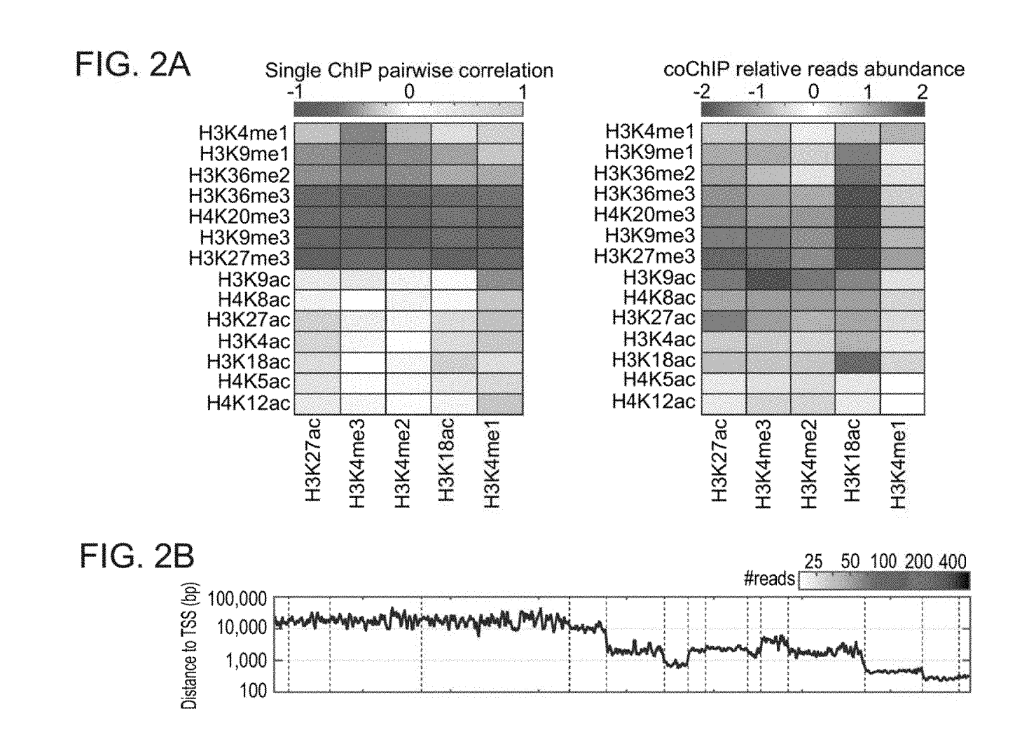

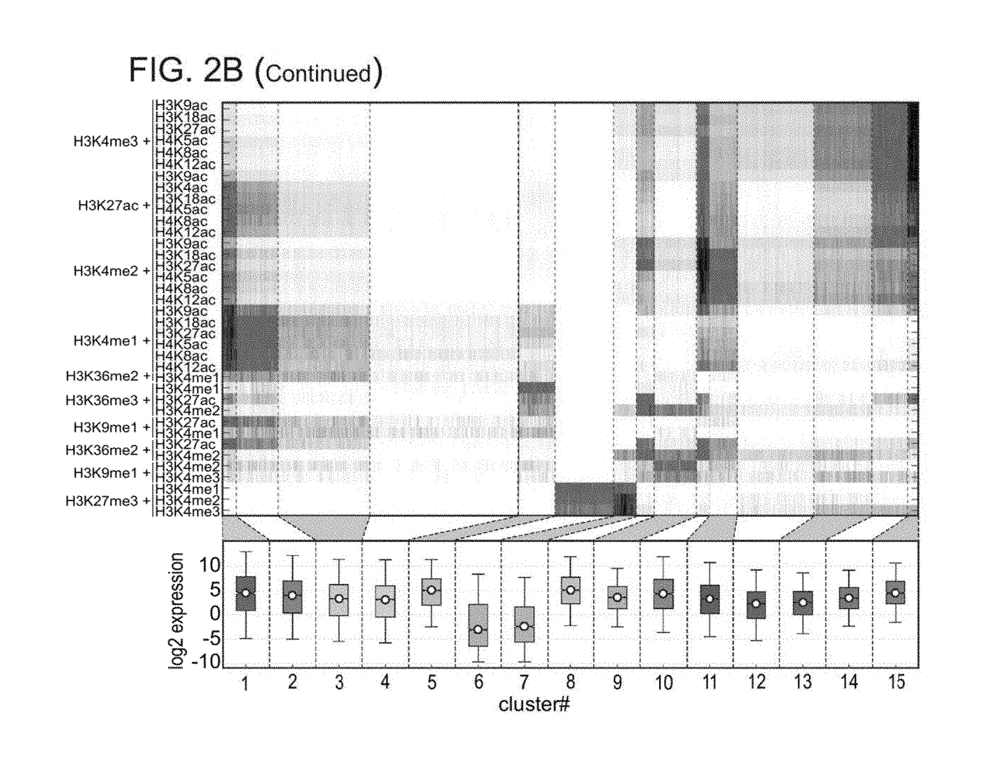

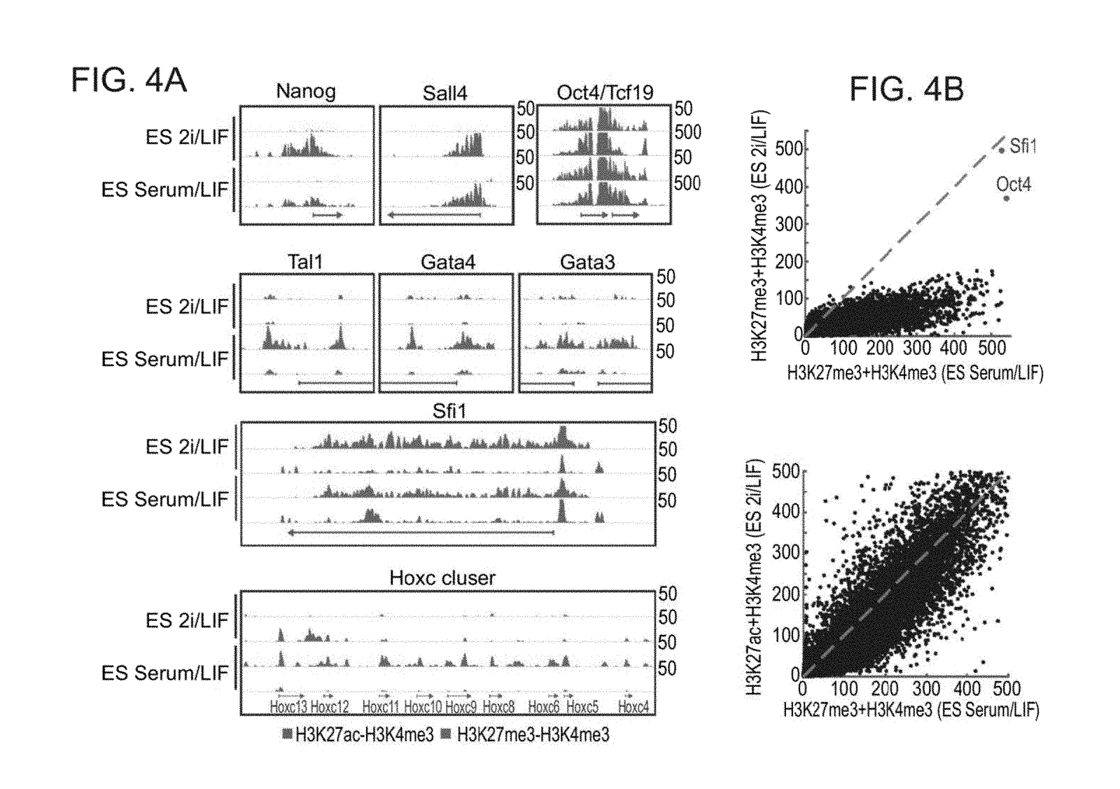

The present inventors have now devised a novel method for combinatorial indexed chromatin immunoprecipitation (Co-ChIP) for probing the interactions between histone marks in a genome-wide manner. The Examples show that Co-ChIP can be applied to profile the co-occurrence of dozens of different histone PTM combinations. In order to investigate the abundance of these interactions, the present inventors used Co-ChIP to profile the co-occurrence of 14 histone PTMs (70 combinations) in primary bone marrow dendritic cells (BMDC), identifying novel antagonistic and synergistic interactions between histone acetylations and H3K4 methylations (FIGS. 2A-B). To further elucidate the interactions of different modifications on the same DNA molecule, the present inventors used Co-ChIP to profile the poised bivalent promoter state (H3K4me3 and H3K27me3) across different tissues and developmental stages. Using this procedure, they determined that bivalent states are dynamic during development and are gained and lost in various mature tissues (FIGS. 4A-B and 5A-C). The novel Co-ChIP protocol presents a new technology to characterize the co-occurrence of pairs of histone PTMs, which can be extensively used to better understand the structure and function of the genome in both health and disease.

Thus, according to a first aspect of the present invention there is provided a method of analyzing DNA molecules in a cell sample, the DNA molecules having DNA binding moiety signatures which are defined by at least two non-identical DNA binding moieties, the method comprising:

(a) labeling DNA molecules of the cell sample with a label that indexes the identity of at least two of the DNA binding moieties of the signatures so as to generate subpopulations of differentially labeled DNA molecules, each subpopulation being in a separate container, wherein the labeling indexes one DNA binding moiety per DNA molecule;

(b) labeling the differentially labeled molecules with another label which indexes the identity of another DNA binding moiety of the signature; and

(c) analyzing the DNA comprising the first label and the second label.

The method of this aspect of the present invention may be used to identify DNA molecules that are bound to pairs of DNA binding moieties. Essentially, DNA molecules are labeled with at least two labels so as to index the identity of the at least two DNA binding moieties.

The labeling of this aspect of the present invention is performed in two separate steps, wherein the first step labels the first DNA binding moiety which is bound to a DNA molecule and the second step labels the second DNA binding moiety which is bound to that DNA molecule.

According to a particular embodiment, the method of this aspect of the present invention is not carried out using a microfluidic device.

Cellular samples may be obtained using methods known in the art. The cells may be obtained from a body fluid (e.g. blood) or a body tissue. The cells may be obtained from a subject (e.g. mammalian subject) or may be part of a cell culture. The cells may arise from a healthy organism, or one that is diseased or suspected of being diseased. According to one embodiment, the sample comprises cells of different cell types (i.e. a heterogeneous population of cells). According to another embodiment, each sample comprises cells of a particular cell type (i.e. a homogeneous population of cells). Samples of a single cell type may be obtained using methods known in the art—for example by FACs sorting. According to still another embodiment, each sample comprises cells from a particular source (e.g. from a particular subject).

According to further embodiments, the sample comprises 100-10,000 cells, 100-5000 cells, 100-2,500 cells, 100-1,000 cells, 100-7,500 cells, 100-5,000 cells, 100-2,500 cells, 100-1000 cells, 100-750 cells, 200-750 cells (for example about 500 cells).

In the cell, (i.e. in the in-vivo environment) from where it is derived, the DNA which is analyzed may be bound permanently or temporarily to the DNA binding moiety.

According to one embodiment, the cells are reversibly crosslinked in order to ensure that DNA binding moieties that are bound to DNA in the in vivo environment (i.e. in the cell) remain bound during the method of this aspect of the present invention. Agents that may be used for reversible cross-linking include but are not limited to formaldehyde or ultraviolet light. Additional agents include, but are not limited to homobifunctional compounds difluoro-2,4-dinitrobenzene (DFDNB), dimethyl pimelimidate (DMP), disuccinimidyl suberate (DSS), the carbodiimide reagent EDC, psoralens including 4,5′,8-trimethylpsoralen, photo-activatable azides such as ¹²⁵I(S-[2-(4-azidosalicylamido)ethylthio]-2-thiopyridine) otherwise known as AET, (N-[4-(p-axidosalicylamido)butyl]-3′[2′-pridyldithio]propionamide) also known as APDP, the chemical cross-linking reagent Ni(II)-NH2-Gly-Gly-His-COOH also known as Ni-GGH, sulfosuccinimidyl 2-[(4-axidosalicyl) amino[ethyl]-1,3-dithiopropionate) also known as SASD, (N-14-(2-hydroxybenzoyl)-N-1 1(4-azidobenzoyl)-9-oxo-8,1 1,14-triaza-4,5-ditheatetradecanoate).

The DNA which is bound to the at least two DNA binding moieties (also referred to herein as the “DNA complexes”) is isolated from the cells. Thus, the present invention contemplates lysing the cells so as to release the complexes from within. Cell lysis may be performed using standard protocols which may be successfully implemented by those skilled in the art including mechanical disruption of cell membranes, such as by repeated freezing and thawing, homogenization, sonication, pressure, or filtration and the use of enzymes and/or detergents (e.g. SDS). For the purposes of chromosomal immunoprecipitation it is important that metal chelators such as EDTA and EGTA as well as protease inhibitors be added to the reaction to prevent degradation of protein DNA complexes.

The phrase “DNA binding moiety signatures” refers to the identity of at least two DNA binding moieties which are bound to the DNA.

As used herein the phrase “DNA binding moiety” refers to an agent or moiety which binds to DNA (in a sequence specific or non-specific manner). The DNA binding moiety may bind to DNA via intercalation, groove binding and/or covalent binding.

In one embodiment, the DNA binding moiety is a DNA binding polypeptide or peptide.

In another embodiment, the DNA binding moiety is a drug (e.g. a small molecule agent).

DNA-binding polypeptides include transcription factors which modulate the process of transcription, various polymerases, nucleases which cleave DNA molecules, and histones which are involved in chromosome packaging and transcription in the cell nucleus. DNA-binding proteins can incorporate such domains as the zinc finger, the helix-turn-helix, and the leucine zipper (among many others) that facilitate binding to nucleic acid.

Exemplary transcription factors include but are not limited to those described in WO 2002014550, the contents of which is incorporated herein by reference.

The following is a list of human histone proteins which may be bound to the analyzed DNA.

TABLE 1

| Super | | | |
|---|---|---|---|
| family | Family | Subfamily | Members |
| Linker | H1 | H1F | H1F0, H1FNT, H1FOO, H1FX |
| | | H1H1 | HIST1H1A, HIST1H1B, HIST1H1C, HIST1H1D, |
| | | | HIST1H1E, HIST1H1T |
| Core | H2A | H2AF | H2AFB1, H2AFB2, H2AFB3, H2AFJ, H2AFV, H2AFX, |
| | | | H2AFY, H2AFY2, H2AFZ |
| | | H2A1 | HIST1H2AA, HIST1H2AB, HIST1H2AC, HIST1H2AD, |
| | | | HIST1H2AE, HIST1H2AG, HIST1H2AI, HIST1H2AJ, |
| | | | HIST1H2AK, HIST1H2AL, HIST1H2AM |
| | | H2A2 | HIST2H2AA3, HIST2H2AC |
| | H2B | H2BF | H2BFM, H2BFS, H2BFWT |
| | | H2B1 | HIST1H2BA, HIST1H2BB, HIST1H2BC, HIST1H2BD, |
| | | | HIST1H2BE, HIST1H2BF, HIST1H2BG, HIST1H2BH, |
| | | | HIST1H2BI, HIST1H2BJ, HIST1H2BK, HIST1H2BL, |
| | | | HIST1H2BM, HIST1H2BN, HIST1H2BO |
| | | H2B2 | HIST2H2BE |
| | H3 | H3A1 | HIST1H3A, HIST1H3B, HIST1H3C, HIST1H3D, |
| | | | HIST1H3E, HIST1H3F, HIST1H3G, HIST1H3H, |
| | | | HIST1H3I, HIST1H3J |
| | | H3A2 | HIST2H3C |
| | | H3A3 | HIST3H3 |
| | H4 | H41 | HIST1H4A, HIST1H4B, HIST1H4C, HIST1H4D, |
| | | | HIST1H4E, HIST1H4F, HIST1H4G, HIST1H4H, |
| | | | HIST1H4I, HIST1H4J, HIST1H4K, HIST1H4L |
| | | H44 | HIST4H4 |

Examples of histone modifications that may be studied include acetylation, methylation, ubiquitylation, phosphorylation and sumoylation. Thus, for example the antibody may bind specifically to H3K4me1, H3K4me2, H3K4me3 or H3K27Ac. Such antibodies are commercially available from a number of sources—for example Abcam.

Examples of covalent post-translationally modified histones which may be bound to the DNA are summarized in Table 2 herein below. Other Examples are provided in the Examples section herein below.

TABLE 2

| Type of | Histone | | | | | | | |
|---|---|---|---|---|---|---|---|---|
| modification | H3K4 | H3K9 | H3K14 | H3K27 | H3K79 | H3K36 | H4K20 | H2BK5 |
| mono- | activation | activation | | activation | activation | | activation | activation |
| methylation | | | | | | | | |
| di-methylation | | repression | | repression | activation | | | |
| tri- | activation | repression | | repression | activation, | activation | | repression |
| methylation | | | | | repression | | | |
| acetylation | | activation | activation | activation | | | | |

The DNA molecules of this aspect of the present invention may be a homogeneous population of molecules, each having the same DNA binding moiety signature. Alternatively, the DNA molecules of this aspect of the present invention are a heterogeneous population of molecules having different DNA binding moiety signatures.

As mentioned, the method of this aspect of the present invention seeks to identify DNAs that are bound to a particular pair of DNA binding proteins. Thus, the DNA molecules of this aspect of the present invention are bound to at least two non-identical DNA binding moieties. In one embodiment, at least one of the two non-identical DNA binding moieties is a modified histone. In another embodiment, both of the two non-identical DNA binding moieties is a modified histone. In yet another embodiment, at least one of the two non-identical DNA binding moieties is a transcription factor. In another embodiment, both of the two non-identical DNA binding moieties is a transcription factor. In still another embodiment, one of the two non-identical DNA binding moieties is a transcription factor and the other of the two non-identical DNA binding moieties is a modified histone.

Exemplary pairs are provided in the Examples section herein below.

According to a particular embodiment, the DNA which is analyzed is no longer than 1000 base pairs, and more preferably no longer than 500 base pairs. If the DNA in the sample is longer, the present invention contemplates a step of shearing or cleaving the DNA. This may be effected by sonication for various amounts of time. The precise time for sonication depends on the cells in the sample and determining the time is within the expertise of one skilled in the art. Examples of sonicators that may be used include the NGS Bioruptor Sonicator (Diagenode) or Branson model 250 sonifier/sonicator as well as restriction enzyme digestion by frequent as well as rare-cutting enzymes including, but not limited to, Ace I, Aci I, Acl I, Afe I, Afl It, Afl El Age I, Ahd I, Alu I, Alw I, AlwN I, Apa I, ApaL I, Apo I, Asc I, Ase I, Ava I, Ava II, Avr II, Bae I, BamH I, Ban I, Ban π, Bbs I, Bbv I, BbvC I, BceA I, Beg I, BciV I, Bel I, Bfa I, BfrB I, Bgl I, Bgl II, Blp I, Bmr I, Bpm I, BsaA I, BsaB I, BsaH I, Bsa I, BsaJ I, BsaW I, BsaX I, BseR I, Bsg I, BsiE I, BsiHKA I, BsiW I, Bsl I, BsmA I, Bs B I, BsmF I, Bsm I, BsoB I, Bspl2861, BspD I, BspE I, BspH I, BspM I, BsrB I, BsrD I, BsrF I, BsrG I, Bsr I, BssH II, BssK I, BssS I, BstAP I, BstB I, BstE II, BstF5 I, BstN I, BstU I, BstX I, BstY I, BstZ171, Bsu361, Btg I, Btr I, Bts I, Cac8 I, Cla I, Dde I, Dpn I, Dpn II, Dra I, Dra HI, Drd I, Eae I, Eag I, Ear I, Eci I, EcoN LEcoO109 I, EcoR I, EcoR V, Fau I, Fnu4H I, Fok I, Fse I, Fsp I, Hae π, Hae I\ddot{υ}, Hga I, Hha I, Hinc II, Hind m, Hinf I, HinP1 I, Hpa I, Hpa II, Hpy188 I, Hpy188 IE, Hpy99 1, HpyCH4iπ, HpyCH4IV, HpyCH4V, Hph I, Kas I, Kpn I, Mbo I, Mbo II, Mfe I, Mlu I, Mly I, Mnl I, Msc I, Mse I, Msl I, MspAl I, Msp I, Mwo I, Nae I, Nar I, Nci I, Nco I, Nde I, NgoM IV, Nhe I, Nla in, Nla IV, Not I, Nru I, Nsi I, Nsp I, Pac I, PaeR7 1, Pci I, PflF I, PflM I, Pie I, Pme I, Pml I, PpuM I, PshA I, Psi I, PspG I, PspOM I, Pst I, Pvu I, Pvu H, Rsa I, Rsr II, Sac I, Sac π, Sal I, Sap I, Sau3A I, Sau96 1, Sbf I, Sea I, ScrF I, SexA I, SfaN I, Sfc I, Sfi I, Sfo, SgrA I, Sma I, Sml I, SnaB I, Spe, Sph I, Ssp I, Stu I, Sty I Swa I, Taq I, Tfi I, Tli I, Tse I, Tsp45 I, Tsp509 I, TspR I, Tthl 11 1, Xba I, Xcm I, Xho I, Xma I and Xmn I.

According to a particular embodiment, the enzyme is not MNase.

Other enzymes that may be used are further described herein below.

The method of this aspect of the present invention comprises a first step of labeling the DNA molecules. The label indexes the identity of one DNA binding moiety of the signature per DNA molecule, and in doing so generates subpopulations of differentially labeled DNA molecules, each subpopulation being in a separate container.

According to one embodiment, the labeling is performed following immobilization of the isolated DNA complexes. Any form of immobilization is conceived by the present inventors as long as it does not interfere with the labeling of the DNA.

According to a particular embodiment, the complexes are not immobilized in microfluidic droplets.

According to one embodiment, the complexes are immobilized on a solid support. Examples of solid supports contemplated by the present invention include, but are not limited to, sepharose, chitin, protein A cross-linked to agarose, protein G cross-linked to agarose, agarose cross-linked to other proteins, ubiquitin cross-linked to agarose, thiophilic resin, protein G cross-linked to agarose, protein L cross-linked to agarose and any support material which allows for an increase in the efficiency of purification of protein/DNA complexes.

According to another embodiment, the complexes are immobilized on a solid support using an antibody that binds to one of the DNA binding moieties of the signature. The antibody of this aspect of the present invention may be polyclonal or monoclonal. The antibodies may bind to the full length proteins as well as against particular epitope amino acid subsets present within those proteins. The antibodies may be of any origin (e.g. rabbit, goat origin, humanized).

Antibodies that recognize histones are commercially available from various sources including for example Abcam and Pierce. For immobilization, the antibodies are attached to a solid support including but not limited to magnetic beads. Other solid phase supports contemplated by the present invention include, but are not limited to, sepharose, chitin, protein A cross-linked to agarose, protein G cross-linked to agarose, agarose cross-linked to other proteins, ubiquitin cross-linked to agarose, thiophilic resin, protein G cross-linked to agarose, protein L cross-linked to agarose and any support material which allows for an increase in the efficiency of purification of protein/DNA complexes.

Methods of attaching antibodies to solid supports are known in the art. For example, linkage of antibodies to solid phase support magnetic beads may be accomplished via standard protocol (Dynal Corporation product information and specifications) and those known and skilled in the art are capable of establishing this linkage successfully. Beads are washed briefly in an appropriate buffer (e.g. phosphate buffered saline (PBS), pH 7.4). About 0.1-1.5 μg of antibody are added per ml of beads, the volume adjusted and the mixture incubated for a suitable length of time (e.g. 12-24 hours at 4° C.). The beads are subsequently collected via a magnet and the supernatant removed. The beads may be washed at least one more time (e.g. in 10 mM Tris-HCl, pH 7.6) for an additional 16-24 hours the bead/antibody complex is ready for immunoprecipitation of protein/DNA complexes.

Magnetic beads contemplated by the present invention include those created by Dynal Corporation such as for example Dynabeads M-450 Tosylactivated (Dynal Corporation). Other Dynabeads M-450 uncoated, Dynabeads M-280 Tosylactivated, Dynabeads M-450 Sheep anti-Mouse IgG, Dynabeads M-450 Goat anti-Mouse IgG, Dynabeads M-450 Sheep anti-Rat IgG, Dynabeads M-450 Rat anti-Mouse IgM, Dynabeads M-280 sheep anti-Mouse IgG, Dynabeads M-280 Sheep anti-Rabbit IgG, Dynabeads M-450 sheep anti-Mouse IgG1, Dynabeads M-450 Rat anti-Mouse IgG1, Dynabeads M-450 Rat anti-Mouse IgG2a, Dynabeads M-450 Rat anti-Mouse IgG2b, Dynabeads M-450 Rat anti-Mouse IgG3. Other magnetic beads which are also contemplated by the present invention as providing utility for the purposes of immunoprecipitation include streptavidin coated Dynabeads.

An alternative method of attaching antibodies to magnetic beads or other solid phase support material contemplated by the present invention is the procedure of chemical cross-linking. Cross-linking of antibodies to beads may be performed by a variety of methods but may involve the utilization of a chemical reagent which facilitates the attachment of the antibody to the bead followed by several neutralization and washing steps to further prepare the antibody coated beads for immunoprecipitation. Yet another method of attaching antibodies to magnetic beads contemplated by the present invention is the procedure of UV cross-linking. A third method of attaching antibodies to magnetic beads contemplated by the present invention is the procedure of enzymatic cross-linking.

A column support fixture rather than beads may be successfully employed for purposes of solid phase. In addition, support fixtures such as Petri dishes, filters, chemically coated test tubes or eppendorf tubes which may have the capability to bind antibody coated beads or other antibody coated solid phase support materials may also be employed by the present invention.

In order to generate two subpopulations of differentially labeled DNA molecules with each subpopulation being in a separate container, the present inventors contemplate performing at least two immunoprecipitation reactions in at least two different containers, each reaction using an antibody which recognizes a different DNA binding moiety. Preferably each of the immunoprecipitation reactions precipitate no more than 20% of the total nucleosomes, more preferably no more than 10% of the total nucleosomes (for example between 0.5-10% of the total nucleosomes or even 0.5-5% of the total nucleosomes).

Thus, complexes of a first aliquot of the sample may be isolated on a solid support using an antibody which specifically binds to a particular DNA binding moiety in a first container; and complexes of a second aliquot of the sample may be isolated on a solid support using an antibody which specifically binds to another DNA binding moiety in a second container.

In one embodiment, at least one of the antibodies binds to a modified histone. In another embodiment, the first antibody binds to a modified histone and the second antibody binds to a non-identical modified histone. In yet another embodiment, at least one of the antibodies binds to a transcription factor. In yet another embodiment, the first antibody binds to a transcription factor and the second antibody binds to a non-identical transcription factor. In still another embodiment, the first antibody binds to a transcription factor and the second antibody binds to a modified histone.

First Labeling of DNA:

The labeling of this aspect of the present invention serves to index the identity of one DNA binding protein that is attached to the DNA. Any labeling technique is contemplated by the present invention including but not limited to end-labeling, labeling of the DNA backbone, sequence specific labeling and sequence non-specific labeling.

According to a particular embodiment, the labeling of this step is effected on the 3′ end of the DNA, on the 5′ end of the DNA or on both ends of the DNA. Labels include fluorescent dyes, quantum dots, magnetic particles, metallic particles, and colored dyes. Generic labels include labels that bind non-specifically to nucleic acids (e.g., intercalating dyes, nucleic acid groove binding dyes, and minor groove binders) or proteins. Examples of intercalating dyes include YOYO-1, TOTO-3, Syber Green, and ethidium bromide.

According to a particular embodiment, the DNA is labeled via a ligation reaction to an adapter that contains a barcode sequence. In one embodiment, the adapter comprises a Solexa adapter. In one embodiment, the adapter comprises an Illumina adapter. The DNA may also be labeled using an enzyme (e.g. Tn5 transposase, Tagment DNA enzyme) that mediates both the fragmentation of double-stranded DNA and ligates synthetic oligonucleotides may be used.

The ligation may be a blunt-ended ligation or using a protruding single stranded sequence (e.g. the sequence may first be A-tailed). The adapter may comprise additional sequences e.g. a sequence recognizable by a PCR primer, sequences which are necessary for attaching to a flow cell surface (P5 and P7 sites), a sequence which encodes for a promoter for an RNA polymerase (as further described herein below) and/or a restriction site. In one embodiment, the adaptor does not comprise sequences which encode a restriction enzyme site. The barcode sequence may be used to identify the identity of the DNA binding moiety. The barcode sequence may be between 3-400 nucleotides, more preferably between 3-200 and even more preferably between 3-100 nucleotides. Thus, the barcode sequence may be 6 nucleotides, 7 nucleotides, 8, nucleotides, nine nucleotides or ten nucleotides. The barcode is typically 4-15 nucleotides.

RNA polymerase promoter sequences are known in the art and include for example a T7 RNA polymerase promoter sequence (e.g. SEQ ID NO: 10 (CGATTGAGGCCGGTAATACGACTCACTATAGGGGC).

For a population of adapters to be used to identify a population of cells, the identification sequence of the adapter differs according to the DNA binding moiety while the rest of the adapter is identical. Since each DNA binding moiety is labeled with an adapter containing a different identification sequence, the nucleic acids which index the DNA binding moieties may be distinguished.

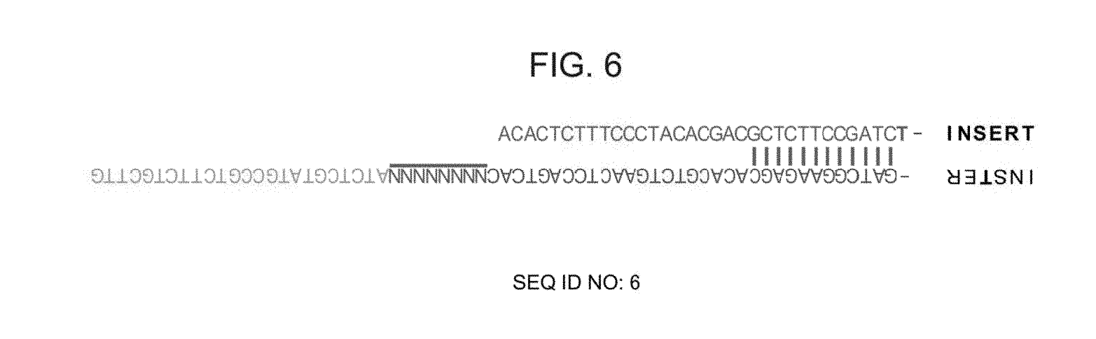

An example of an adaptor that may be used according to embodiments of this aspect of the present invention is illustrated in FIG. 6.

Removal of non-ligated adaptors may be effected using any method known in the art (for example 10 mM TrisCl). The buffer for removal of non-ligated adaptors may also comprise protease inhibitors.

If the complexes were immobilized prior to the labeling stage, the next stage comprises release of the immobilized complexes. The present inventors contemplate any method of releasing the complexes so long as the DNA of the complexes remains labeled. It will be appreciated that more than one round of release may be performed (for example, two rounds of release). Methods include use of detergents (e.g. DTT, Sodium Deoxycholate, SDS), high salt (e.g. 200-2000 mM, e.g. 500 mM salt, NaCl) and/or heat (e.g. about 37° C.). An exemplary concentration of SDS is 2%. An exemplary concentration of NaCl is 1 molar. Protease inhibitors may also be included in the buffer. Preferably, the method used releases more than 40% of the complexes, more preferably more than 50% of the complexes, and even more preferably more than 60% of the complexes.

Following the labeling, subpopulations of differentially labeled molecules are generated, each in a separate container. Thus, in one container there is a subpopulation of molecules which are indexed as being bound to DNA binding moiety (1), and in another container there is a subpopulation of molecules which is indexed as being bound to DNA binding moiety (2).

Optionally, the DNA is then released from the immobilizing agent.

The DNA aliquots (either together with the immobilizing agent or released from the immobilizing agent) may then be pooled. The complexes may be purified at this stage and/or concentrated. This may be effected using any method known in the art including ultracentrifugation (e.g. using a centricon with a 50 kDa cutoff). Additionally, or alternatively, the complexes may be washed prior to the next stage to ensure that the complexes are capable of binding to an additional antibody. For example, the final salt and detergent level should be compatible with antibody integrity. Thus, for example, the detergent level should be less than about 1 mM and the salt concentration (for example NaCl) should be less than about 150 mM. An exemplary buffer which may be used to incubate the complexes is as follows: 10 mM Tris-HCl pH 8.0, 140 mM NaCl, 1% Triton X-100, 0.1% SDS, 0.1% DOC, 1 mM EDTA, 1× Protease Inhibitors.

Second Labeling

The second labeling (or tagging) step of this aspect of the present invention labels the differentially labeled molecules with another label which indexes the identity of another DNA binding moiety of the signature.

As in the first labeling step, the second labeling (or tagging step) may label the 3′ end of the DNA, the 5′ end of the DNA or on both ends of the DNA. According to a particular embodiment, the first labeling step labels the 5′ end of the DNA and the second labeling step labels the 3′ end of the DNA.

Exemplary labels are described herein above.

In a particular embodiment, the first labeling step labels one end of the DNA and the second labeling step indexes the other end of the DNA.

According to another embodiment, the first labeling step labels one end of the DNA via a ligation reaction to an adapter that contains a barcode sequence that indexes the first DNA binding moiety and the second labeling step indexes the other end of the DNA via a ligation reaction to an adapter that contains a barcode sequence that indexes the second DNA binding moiety, a PCR reaction or an in vitro transcription reaction as further described herein below. Exemplary primer sequences are described herein below.

As in the first labeling step, the second labeling step may also be performed on subpopulations of differentially labeled DNA molecules. Thus, the present inventors contemplate performing at least two immunoprecipitation reactions in at least two different containers prior to the second labeling step, each reaction using an antibody which recognizes a different DNA binding moiety.

Methods of immunoprecipitating the complexes and exemplary antibodies that may be used for same are described herein above.

Thus, complexes of a first aliquot of the pooled differentially labeled DNA molecules may be isolated on a solid support using an antibody which specifically binds to a particular DNA binding moiety in a first container; and complexes of a second aliquot of the pooled differentially labeled DNA molecules may be isolated on a solid support using a different antibody which specifically binds to another DNA binding moiety in a second container.

In one embodiment, at least one of the antibodies binds to a modified histone. In another embodiment, the first antibody binds to a modified histone and the second antibody binds to a non-identical modified histone. In yet another embodiment, at least one of the antibodies binds to a transcription factor. In yet another embodiment, the first antibody binds to a transcription factor and the second antibody binds to a non-identical transcription factor. In still another embodiment, the first antibody binds to a transcription factor and the second antibody binds to a modified histone.

It will be appreciated that the second labeling step may be effected on crosslinked DNA or on reverse crosslinked DNA.

The complexes may be reverse cross-linked so as to release the DNA fragments prior to the analysis. This may be effected in one round or more than one round. Those known and skilled in the art are capable of successfully reversing cross-linkages via conventional chromosomal immunoprecipitation protocols. Reversal of cross-linkages is accomplished through an incubation of the isolated protein/DNA complexes at high temperatures, preferably above 50° C. for at least 6 hours, (e.g. 65° C. for about 8 hours). Proteinase K treatment may also be effected at this stage. It is contemplated by the present invention that reversal of cross-linkages through chemical methods such as alkali treatment as well as UV or enzymatic manipulation may be implemented successfully and are covered by the presently described invention for the purposes of the present invention, as long as the DNA of the complex is not altered in any way such that it cannot undergo sequence analysis.

The present invention further contemplates amplifying the DNA (e.g. by PCR) following the reverse crosslinking. As mentioned, the second label may be attached to the DNA during the amplification process.

In order to increase sensitivity, the released (reverse crosslinked) DNA may undergo a stage of in vitro transcription (according to this embodiment, the adaptor sequence in the labeling stage should comprise an RNA polymerase binding site, as further described herein above). The DNA is incubated with an RNA polymerase (e.g. T7), ribonucleotide triphosphates, preferably in a buffer system that includes DTT and magnesium ions. The sample is then incubated with a DNAse to remove the DNA from the sample.

As mentioned, the second label may be attached to the DNA during the in vitro transcription process.

For further enhancement of sensitivity, an additional step may be carried out to ensure that both ends of the molecule are bar-coded. Thus, the present invention contemplates ligating another sequencing adaptor to the in-vitro synthesized RNA molecules using an RNA ligase enzyme (e.g. T4 RNA ligase). An exemplary buffer for performing this reaction is as follows: 9.5% DMSO, 1 mM ATP, 20% PEG8000 and 1 U/μ 1 T4 ligase in 50 mM Tris HCl pH7.5, 10 mM MgCl2 and 1 mM DTT.

Reverse transcription may then be carried out to convert the synthesized RNA into DNA. An exemplary reverse transcriptase enzyme is the Affinity Script RT enzyme (commercially available from Agilent). An exemplary reaction mix may contain a suitable buffer supplemented DTT, dNTPs, the RT enzyme and a primer complementary to the ligated adapter.

The DNA may be sequenced using any method known in the art—e.g. massively parallel DNA sequencing, sequencing-by-synthesis, sequencing-by-ligation, 454 pyrosequencing, cluster amplification, bridge amplification, and PCR amplification, although preferably, the method comprises a high throughput sequencing method. Typical methods include the sequencing technology and analytical instrumentation offered by Roche 454 Life Sciences™, Branford, Conn., which is sometimes referred to herein as “454 technology” or “454 sequencing.”; the sequencing technology and analytical instrumentation offered by Illumina, Inc, San Diego, Calif. (their Solexa Sequencing technology is sometimes referred to herein as the “Solexa method” or “Solexa technology”); or the sequencing technology and analytical instrumentation offered by ABI, Applied Biosystems, Indianapolis, Ind., which is sometimes referred to herein as the ABI-SOLiD™ platform or methodology.

Other known methods for sequencing include, for example, those described in: Sanger, F. et al., Proc. Natl. Acad. Sci. U.S.A. 75, 5463-5467 (1977); Maxam, A. M. & Gilbert, W. Proc Natl Acad Sci USA 74, 560-564 (1977); Ronaghi, M. et al., Science 281, 363, 365 (1998); Lysov, 1. et al., Dokl Akad Nauk SSSR 303, 1508-1511 (1988); Bains W. & Smith G. C. J. Theor Biol 135, 303-307 (1988); Drnanac, R. et al., Genomics 4, 114-128 (1989); Khrapko, K. R. et al., FEBS Lett 256.118-122 (1989); Pevzner P. A. J Biomol Struct Dyn 7, 63-73 (1989); and Southern, E. M. et al., Genomics 13, 1008-1017 (1992). Pyrophosphate-based sequencing reaction as described, e.g., in U.S. Pat. Nos. 6,274,320, 6,258,568 and 6,210,891, may also be used.

Following sequencing, the DNA may be aligned with genomes, e.g., to determine which portions of the genome were epigenetically modified, e.g., via methylation. Analysis of the sequences may provides information relating to potential transcription factor binding sites and/or epigenetic profiling, as further described in the Examples section herein below.

Kits

Any of the compositions described herein may be comprised in a kit. In a non-limiting example the kit comprises the following components, each component being in a suitable container:

(i) at least one antibody which specifically binds to a transcription factor;
(ii) at least one antibody which specifically binds to a post-translationally modified histone; and
(iii) a DNA labeling agent (e.g. the adaptors which comprise the barcode sequences as described herein above).

The kit may comprise additional components including, but not limited to an RNA polymerase, a DNAse and/or a reverse transcriptase. Additional components include a crosslinker, a protease enzyme, nucleotide triphosphates and/or a ligase. The kit may also comprise the appropriate buffers for carrying out the immunoprecipitation procedure described herein. Exemplary buffers are described herein above and in the Examples section herein below. Exemplary antibodies that may be included in the kit are described herein above.

The kit may further comprise an antibody which immobilizes at least 50% of the chromatin of a cell. Thus, for example the antibody may specifically bind to an H2, H3 or H4 histone. According to a particular embodiment the antibody specifically binds to H3.

According to particular embodiment, the kits of this aspect of the present invention do not comprise MNAse.

The containers of the kits will generally include at least one vial, test tube, flask, bottle, syringe or other containers, into which a component may be placed, and preferably, suitably aliquoted. Where there is more than one component in the kit, the kit also will generally contain a second, third or other additional container into which the additional components may be separately placed. However, various combinations of components may be comprised in a container.

When the components of the kit are provided in one or more liquid solutions, the liquid solution can be an aqueous solution. However, the components of the kit may be provided as dried powder(s). When reagents and/or components are provided as a dry powder, the powder can be reconstituted by the addition of a suitable solvent.

A kit will preferably include instructions for employing, the kit components as well the use of any other reagent not included in the kit. Instructions may include variations that can be implemented.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N.Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### MATERIALS AND METHODS

Co-ChIP Protocol

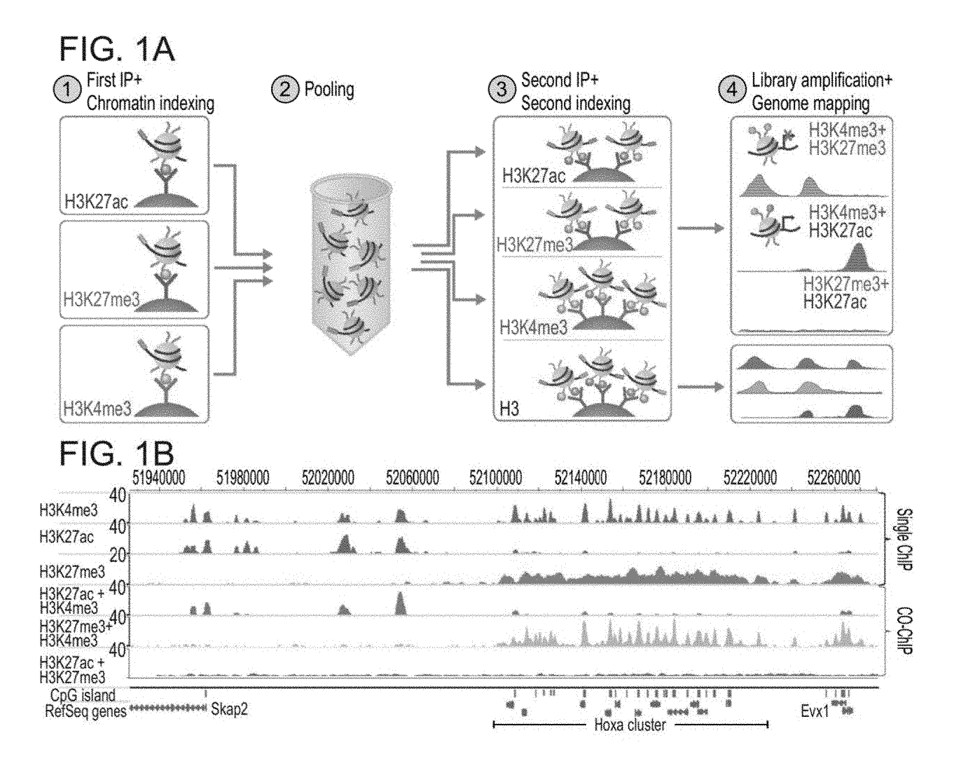

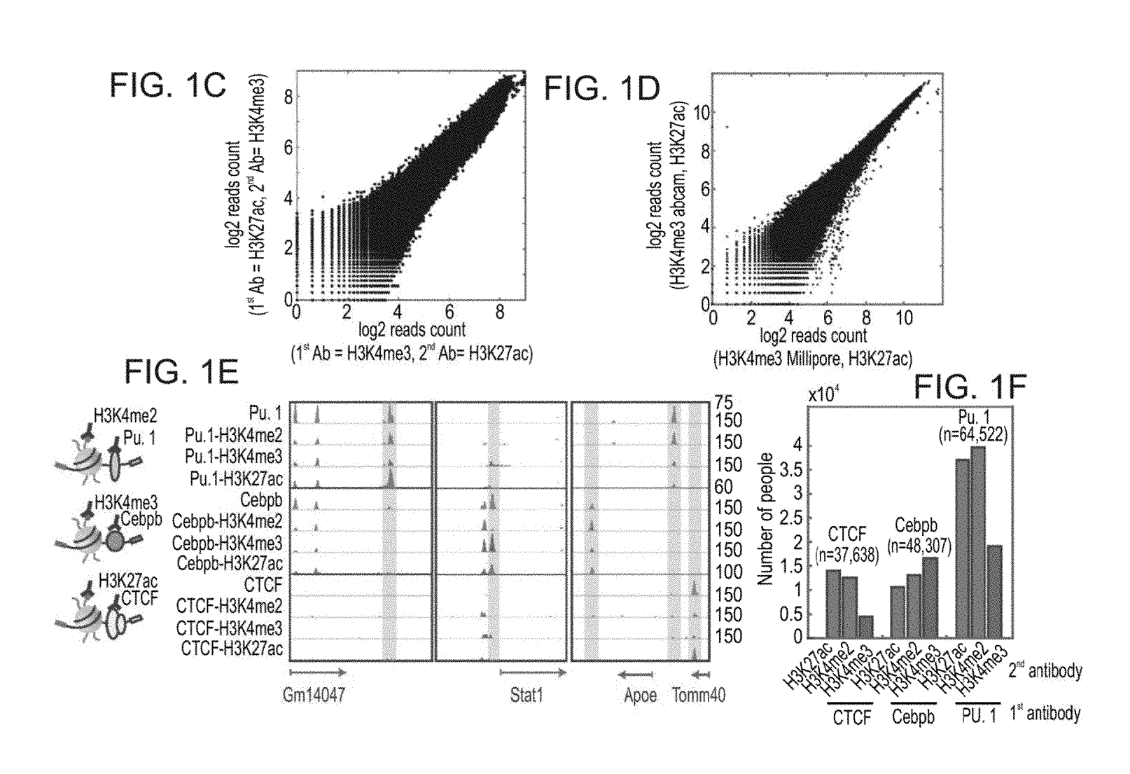

CoChIP is a modular protocol (FIG. 1A) in which every step has been optimized to minimize noise. 1) Cells are cross-linked and frozen in aliquots of 10-20 million cells; 2) chromatin is sheared, 3) Immobilized on specific antibody coated magnetic beads, 4) Bead immobilized chromatin is indexed by ligation of sequencing adaptors. 5) The indexed chromatin is released from the antibody coated magnetic beads using antibody denaturing conditions and pooled together with other samples in a single tube, 6) The chromatin pool is washed to remove antibody denaturing elements. 7) The chromatin pool is subjected to a second chromatin immunoprecipitation step 8) RNA and proteins are degraded and DNA is reverse crosslinked. 9) ChIPed DNA, which contains P5 and P7 Illumina sequences, is purified using SPRI AMPure XP beads. 10) DNA is amplified by PCR.

I. Cell Crosslinking and Harvesting

Cells (both ES and BMDCs) growing on 10 cm plates are crosslinked by adding formaldehyde to a final concentration of 1% and incubated at room temperature for 8 min with moderate shaking. Immediately after, glycine is added to a final concentration of 125 mM and incubated for 5 minutes at room temperature to stop the crosslinking by quenching the free formaldehyde. Then, cells are scraped and transfer to a 50 ml tube at 4° C.; cells are pelleted by centrifugation at 300×G/4° C. for 10 min and washed 3 times with 10 ml of ice-cold PBS/5 mM EDTA; finally, crosslinked cells are re-suspended in 1 ml of PBS/5 mM EDTA is supplemented with protease inhibitors (Roche) and pelleted by centrifugation at 500×G, the supernatant is removed and crosslinked cell pellets are snap frozen and stored at −80° C.

2. Sonication

10-20 million cell aliquots are thawed on ice and re-suspended in 750 μl of RIPA-I. Then, cells are sonicated using a Branson tip sonicator with pulses of 0.7 seconds ON/1.3 seconds OFF using amplitude equivalent to 12 watts per pulse; the cells are kept cold during sonication using a cooler set at −4° C. To achieve a composition of >90% nucleosomes, cells are sonicated using the mentioned conditions for 3 periods of 3 minutes each, keeping the cells for 5 minutes on ice between each period. After sonication 750 μl of RIPA-II are added to the cells, the solution is mixed by vortexing and centrifuged at >14.000 xG/4° C. for 10 min to pellet the insoluble cell debris. Cleared chromatin extracts are transferred to clean tubes and can be stored in single use aliquots at −80° C.

3. First IP

Chromatin extracts are immunoprecipitated with the relevant antibody in 300 μl of RIPA buffer; the relevant amount of chromatin is added supplemented with RIPA buffer is to reach a final volume of 300 μl. The cell and antibody amounts as well as the incubation time for each antibody were calibrated to produce high specific and efficient IPs and are listed below:

H3K4me1 (ab8895): 2 μg antibody; 0.5 million cells; 3 h
H3K4me2 (ab32356): 4 μg antibody; 0.5 million cells; 3 h
H3K4me3 (Millipore, 07-473): 2.5 μg antibody; 0.5 million cells; 3 h
H3K9me1 (Abcam): 4 μg antibody; 0.7 million cells; 8 h
H3K27me3 (Millipore) 4 μg antibody; 0.5 million cells; 3 h
H3K36me2: 2.5 μg antibody; 0.5 million cells; 8 h
H3K36me3: 2.5 μg antibody; 0.5 million cells; 8 h
H3K4ac: 3 μg antibody; 0.7 million cells; 8 h
H3K9ac: 4 μg antibody; 1 million cells; 8 h
H3K18ac: 3 μg antibody; 0.7 million cells; 5 h
H3K27ac: (ab4729): 3 μg antibody; 0.7 million cells; 5 h
H4K5ac: 4 μg antibody; 1 million cells; 8 h
H4K8ac: 4 μg antibody; 1 million cells; 8 h
H4K12ac: 4 μg antibody; 1 million cells; 8 h

After the incubation with the relevant antibody, 40 μl of Protein G beads (prewashed and re-suspended in RIPA+Pi) are added to each IP tube and incubated for 1 h at 4° C. in order to capture the immuno-complexes.

For transcription factors Co-ChIP, 10 million cells were used performing an IP of 9 h at 4° C. 10 μg of TF antibody pre-coupled to 50 μl of Protein G beads were used. The antibodies used were: anti PU.1 (Santa Cruz, sc352-x), anti-Cebpb, anti-CTCF. In order to couple the Beads to the TF antibody, beads were washed once (200 μl) in a binding/blocking buffer (PBS, 0.5% Tween 20, 0.5% BSA), incubated with 10 μg of antibody in binding/blocking buffer for 1 hour at room temperature, and then washed to remove excess antibody.

For every histone mark/TF, the first IP was performed using at least three independent replicates.

4. Washes-I:

A 96 well magnet was used (Invitrogen) in all further steps and washes were performed on ice. First, samples are magnetized in a 1.5 ml magnet, supernatant is removed and beads are re-suspended in 200 μl of RIPa-Pi buffer and transferred to an ice-cold 96 well plate. Then, samples are washed 5 times with cold RIPA+Pi (200 μl per wash), 3 times with RIPA500+Pi (200 μl per wash), 3 times with LiCl buffer+Pi (10 mM TE, and 4 times with Tris-Pi pH 7.5. After the last wash, the beads were re-suspended in 22.5 μl of ice-cold Tris-Pi pH 7.5 buffer and put on ice until the next step.

5. Chromatin Indexing:

Magnet based bead capture was used to efficiently add, wash and remove the different master mixes used in the indexing process. All the reactions were done while chromatin was bound to the antibody coated magnetic beads. First, Chromatin End Repair was performed by adding 27.5 μl of a master mix: 25 μl 2×ER mix, 2 μl T4 PNK enzyme (10 U/ul NEB), and 0.5 μl T4 polymerase (3 U/ul NEB) to each well and mixing thoroughly by pipetting.

Samples were incubated in a thermal cycler at 12° C./25 min and 25° C./25 min. After end repair, bead bound chromatin was magnetized on an ice-cold magnet, washed once with 150 μl of ice-cold Tris-Pi pH 8 and re-suspended in 40 μl of the same buffer. Chromatin was A-tailed by adding 20 μl of a master mix (17 μl A-base add mix, 3 μl Klenow (3′->5′ exonuclease, 3 U/ul, NEB) to each well; samples were thoroughly mixed and incubated at 37° C. for 30 min in a thermal cycler. After end repair, bead bound chromatin was magnetized on an ice-cold magnet, washed once with 150 μl of ice-cold Tris-Pi pH 8 and re-suspended in 18 μl of the same buffer. Finally, the bead-bound chromatin was indexed by adding to each well, 5 μl of 1 μM Y-Shaped Indexed Adaptors plus 34 μl of AL master mix (29 μl 2× Quick Ligation Buffer and 5 μl Quick DNA ligase (NEB). Samples were thoroughly mixed and incubated at 25° C. for 40 min in a thermal cycler. After chromatin indexing, bead bound indexed chromatin was magnetized on an ice-cold magnet and washed once with 150 μl of ice-cold Tris-Pi pH 8 in order to remove the free adaptors. After this wash, Tris-Pi buffer was removed and the beads containing no buffer were stored on ice until the next step.

6. Chromatin Release

Denaturing conditions (DTT, high salt and detergent) and heat were used to release the indexed chromatin from the antibody coated magnetic beads. Right after the post-Indexing wash, samples were taken out of the magnet; beads were re-suspended in 15 μl of 100 mM DTT and incubated for 5 min at Room Temp. Then, 15 μl of Chromatin Release Buffer were added to each well, samples were mixed thoroughly and incubated at 37° C. for 30 min.

After the release incubation, magnetic beads were re-suspended and pooled together in groups of 24 samples resulting in a pool volume of 720 μl. The pool of indexed chromatin samples was magnetized to retrieve the free indexed chromatin from the magnetic beads and diluted 1 to 20 in 10 mM Tris Cl, 100 mM NaCl, 1 mM EDTA+Protease Inhibitors. The diluted pool was mixed by vortexing and centrifuged at >3000×G/20° C. for 10 min to precipitate the beads. The diluted pool was concentrated using two 50 kDa cutoff Centricon (Amicon), one half of the pool was added to each centricon and the volume was filled to 15 ml with Centricon buffer, then the centricons are centrifuged at 1500×G/20° C. for 15 min and the concentrated chromatin (roughly 150 μl) is transferred to a clean tube. Finally, to each sample, 1 volume of Centricon Equilibration Buffer is added. If the pool is going to be subjected to more than one second IP, 300 μl of RIPA-Pi buffer per secondary IP are added. Before setting up the second IP, the chromatin pool is spun down >12.000 xG/4° C. for 5 min to pellet any debris present and transferred to clean tubes. Concentrated indexed chromatin pools can be stored at −80° C.

7. Second IP

Indexed chromatin pools are always immunoprecipitated with the relevant antibody in a 300 ul reaction. The amount and clone of secondary antibodies is listed below. Immunoprecipitation is performed for 3 h at 4° C. followed by 1 h incubation with 40 μl of prewashed Protein G beads (resuspended in RiPA-Pi buffer), which capture the immunocomplexes.

H3K4me1 (ab8895): 2 μg antibody
H3K4me2 (ab32356): 4 μl antibody
H3K4me3 (Millipore, 07-473): 2.5 μg antibody
H3K27me3 (Millipore) 4 μg antibody
H3K9me1 (ab), H3K36me2, H3K36me3: 4 μg antibody, here the IP is performed for 6 h.

8. Washes and ChIPed DNA Elution

A 96 well magnet was used (Invitrogen) in all further steps. Samples are magnetized in a 1.5 ml magnet and beads are re-suspended in 200 μl of RIPa-Pi buffer and transferred to an ice-cold 96 well plate. Then, samples are washed 5 times with cold RIPA (200 μl per wash), 3 times with RIPA-500 buffer (200 μl per wash), 3 times with LiCl buffer, twice with TE, and then eluted in 50 μl of ChIP elution buffer. The eluate was treated sequentially with 2 μl of RNaseA (Roche, 11119915001) for 30 min at 37° C., 2.5 μl of Proteinase K (NEB, P8102S) for 1 hour at 55° C. and 8 hours at 65° C. to revert formaldehyde crosslinking.

9. CoChIPed DNA isolation.

SPRI cleanup steps were performed using 96 well plates and magnets. 90 μl SPRI were added to the reverse-crosslinked samples, pipette-mixed 15 times and incubated for 6 minutes. Supernatant were separated from the beads using a 96-well magnet for 5 minutes. Beads were washed on the magnet with 70% ethanol and then air dried for 5 minutes. The DNA was eluted in 23 μl EB buffer (10 mM Tris-HCl pH 8.0) by pipette mixing 25 times.

10. Library Amplification and Sequencing.

The library was completed through 12 cycles of PCR (98 C/20 sec, 55 C/30 sec, 72 C/45 sec) using 0.5 μM of PCR forward and PCR reverse primers and PCR ready mix (Kapa Biosystems). The forward primer is different for each secondary antibody used and contains “i5 barcoded” Illumina P5-Read1 sequences; the reverse primer is unique and contains the P7-Read2 sequences. The amplified pooled single-cell library was purified with 1× volumes of SPRI beads. Library concentration was measured with a Qubit fluorometer (Life Technologies) and mean molecule size was determined with a 2200 TapeStation instrument (Agilent Technologies). coChIP libraries were sequenced using an Illumina HiSeq 1500.

Buffers

TABLE 3
LB1

| | Stock | Final | For 100 ml | For 250 ml |
|---|---|---|---|---|
| Hepes-KOH | 1M | 50 mM | 5 ml | 12.5 ml |
| EDTA, pH 8.0 | 0.5M | 1 mM | 0.2 ml | 0.5 ml |
| NaCl | 5M | 140 mM | 2.8 ml | 7 ml |
| Triton x-100 | 10% | 0.25% | 2.5 ml | 6.25 ml |
| NP-40 | 10% | 0.5% | 5 ml | 12.5 ml |
| Glycerol | 100% | 10% | 10 ml | 25 ml |
| H₂O | | | 74.5 ml | 186.25 ml |

TABLE 4
RIPA:

| | Stock | Final | For 100 ml | For 250 ml |
|---|---|---|---|---|
| Tris-HCl, pH | 1M | 10 mM | 1 ml of | 2.5 ml of |
| 8.0 | | | 100xTE | 100xTE |
| EDTA, pH | 100 mM | 1 mM | | |
| 8.0 | | | | |
| NaCl | 5M | 140 mM | 2.8 | 7 ml |
| Triton x-100 | 10% | 1% | 10 ml | 25 ml |
| SDS | 10% | 0.1% | 1 ml | 2.5 ml |
| DOC | 5% | 0.1% | 2 ml | 5 ml |
| H₂O | | | 83.2 ml | 208 |

TABLE 5
RIPA I (double SDS, no Triton):

| | Stock | Final | For 100 ml | For 250 ml |
|---|---|---|---|---|
| Tris-HCl, pH | 1M | 10 mM | 1 ml of | 2.5 ml of |
| 8.0 | | | 100xTE | 100xTE |
| EDTA, pH | 100 mM | 1 mM | | |
| 8.0 | | | | |
| NaCl | 5M | 140 mM | 2.8 ml | 7 ml |
| SDS | 10% | 0.2% | 2 ml | 5 ml |
| DOC | 5% | 0.1% | 2 ml | 5 ml |
| H₂O | | | 92.2 ml | 230.5 |

*Keep at room temperature.

TABLE 6
RIPA II (no SDS, double Triton):

| | Stock | Final | For 100 ml | For 250 ml |
|---|---|---|---|---|
| Tris-HCl, pH | 1M | 10 mM | 1 ml of | 2.5 ml of |
| 8.0 | | | 100xTE | 100xTE |
| EDTA, pH | 100 mM | 1 mM | | |
| 8.0 | | | | |
| NaCl | 5M | 140 mM | 2.8 ml | 7 ml |
| Triton x-100 | 10% | 2% | 20 ml | 50 ml |
| DOC | 5% | 0.1% | 2 ml | 5 ml |
| H₂O | | | 74.2 ml | 185.5 ml |

TABLE 7
End Repair 2X. Aliquot and store at −20° C.

| | Add for 10 ml | 2X conc | 1X conc |
|---|---|---|---|
| 1M TrisCl ph 7.5 | 1 ml | 100 mM | 50 mM |
| 1M MgCl2 | 200 ul | 20 mM | 10 mM |
| 500 mM DTT | 400 ul | 20 mM | 10 mM |
| 10 mM ATP | 2000 ul | 2 mM | 1 mM |
| 10 mM dNTP (each) | 800 ul | 0.8 mM | 0.4 mM |
| H₂O | 5400 ul | | |

dA-Mix Buffer. Aliquot and store at −20° C.

5940 ul NEB buffer 2 10×

99 ul dATP 100 mM

10791 ul H₂O

TABLE 8
2X Chromatin Release Buffer (make fresh)

| | 2X | Stock | Add for 1 ml |
|---|---|---|---|
| NaCl | 1000 mM | 5M | 200 ul |
| Deoxycholate | 2% | 5% | 400 ul |
| SDS | 4% | 20% | 200 ul |
| Complete Mini EDTA-Roche | 2X | 50X | 40 ul |
| H₂O | | | 160 ul |

TABLE 9
Dilution Buffer (Store at RT)

| | 1X | Stock | Add for 100 ml |
|---|---|---|---|
| Tris Cl | 10 mM | 1M | 1 ml |
| NaCl | 100 mM | 5M | 2 ml |
| EDTA | 1 mM | 500 mM | 200 ul |
| H₂O | | | 97.8 ml |
| Complete Mini EDTA- | 1X | | |

TABLE 10
Centricon Buffer (Store at Room Temp)

| | 1X | Stock | Add for 500 ml |
|---|---|---|---|
| Tris Cl | 10 mM | 1M | 5 ml |
| NaCl | 140 mM | 5M | 14 ml |
| EDTA | 1 mM | 500 mM | 1 ml |
| SDS | 0.1% | 20% | 2.5 ml |
| Na-Deoxycholate | 0.1% | 5% | 10 ml |
| H₂O | | | 97.8 ml |

TABLE 11
Centricon Equilibration Buffer (store at 4° C.)

| | 1X | Stock | Add for 50 ml |
|---|---|---|---|
| Tris Cl | 10 mM | 1M | 500 ul |
| NaCl | 140 mM | 5M | 1.4 ml |
| EDTA | 1 mM | 500 mM | 100 ul |
| SDS | 0.1% | 20% | 250 ul |
| Na-Deoxycholate | 0.1% | 5% | 1 ml |
| Tx-100 | 2% | 10% | 10 ml |
| H₂O | | | 36.75 ml |
| Complete Mini Roche | 2X | | |

TABLE 12
RIPA-500 (store at 4° C.):

| | Stock | Final | For 100 ml | For 250 ml |
|---|---|---|---|---|
| Tris-HCl, | 1M | 10 mM | 1 ml of 100xTE | 2.5 ml of |
| pH 8.0 | | | | 100xTE |
| EDTA, pH 8.0 | 100 mM | 1 mM | | |
| NaCl | 5M | 500 mM | 10 ml | 25 ml |
| Triton x-100 | 10% | 1% | 10 ml | 25 ml |
| SDS | 10% | 0.1% | 1 ml | 2.5 ml |
| DOC | 5% | 0.1% | 2 ml | 5 ml |
| H₂O | | | 76 ml | 190 ml |

TABLE 13
LiCl wash buffer (store at 4° C.):

| | Stock | Final | For 100 ml | For 250 ml |
|---|---|---|---|---|
| Tris-HCl, | 1M | 10 mM | 1 ml of 100xTE | 2.5 ml of |
| pH 8.0 | | | | 100x TE |
| EDTA, pH 8.0 | 100 mM | 1 mM | | |
| LiCl | 8M | 250 mM | 3.125 ml | 7.81 ml |
| NP-40 | 100% | 0.5% | 0.5 ml | 1.25 ml |
| DOC | 5% | 0.5% | 10 ml | 25 ml |
| H₂O | | | 85.37 ml | 213.4 ml |

TABLE 14
1xTE (store at 4° C.):

| | Stock | Final | For 100 ml | For 250 ml |
|---|---|---|---|---|
| Tris-HCl | 1M | 10 mM | 1 ml of 100xTE | 2.5 ml of |
| pH 8.0 | | | | 100xTE |
| EDTA pH 8.0 | 100 mM | 1 mM | | |
| H₂O | | | 99 ml | 247.5 ml |

TABLE 15
5% Na-deoxycholate (DOC) (Store at RT):

| | Stock | Final | For 100 ML | For 250 ml |
|---|---|---|---|---|
| Na-Deoxycholate | powder | 5% (weight/ | 5 gr | 25 gr |
| H₂O | | volume) | 100 ml | 250 ml (final) |
| | | | (final) | |

TABLE 16
ChIP elution buffer (store at Room Temp):

| | Stock | Final | For 100 ml | For 250 ml |
|---|---|---|---|---|
| Tris-HCl, | 1M | 10 mM | 1 ml of 100xTE | 2.5 ml of 100xTE |
| pH 8.0 | | | | |
| EDTA, | 0.5M | 5 mM | 0.8 ml | 2 ml |
| pH 8.0 | | | | |
| NaCl | 5M | 300 mM | 6 ml | 15 ml |
| SDS | 10% | 0.3% | 3 ml | 12.5 ml |
| H₂O | | | 88.2 ml | 218 ml |

ChIP Adaptors:

Universal ChIP Adaptor:

(SEQ ID NO: 1)
ACACTCTTTCCCTACACGACGCTCTTCCGATC*T
* indicates phosphorothioate

Sequence of entire Read1
12 bp complementary with i7, this serves to make asymmetric Y-shaped adaptors

Indexed Adaptors with 5′ Phosphorylation
Barcode
i7: i7 reads index in “forward”=Read2 in reverse complementary
P7: Attaches to the Illumina's flow cell

A1 Index
(SEQ ID NO: 2)
/5Phos/GATCGGAAGAGCACACGTCTGAACTCCAGTCACCTACCAG
GATCTCGTATGCCGTCTTCTGCTTG
B1
(SEQ ID NO: 3)
/5Phos/GATCGGAAGAGCACACGTCTGAACTCCAGTCACCATGCTT
AATCTCGTATGCCGTCTTCTGCTTG
C1
(SEQ ID NO: 4)
/5Phos/GATCGGAAGAGCACACGTCTGAACTCCAGTCACGCACATC
TATCTCGTATGCCGTCTTCTGCTTG
D1
(SEQ ID NO: 5)
/5Phos/GATCGGAAGAGCACACGTCTGAACTCCAGTCACTGCTCGA
CATCTCGTATGCCGTCTTCTGCTTG

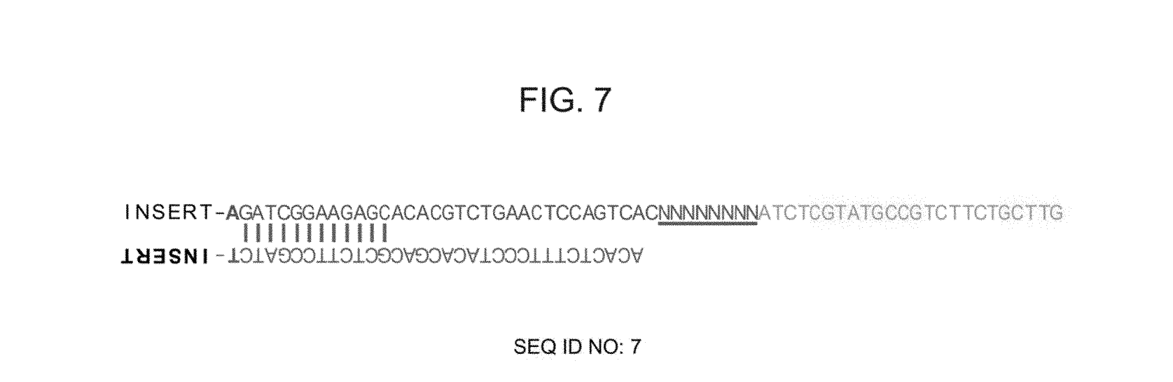

Examples of 3′ and 5′ adapters are illustrated in FIGS. 6 and 7.

PCR Enrichment Primers:

Primer1:
(SEQ ID NO: 8)
5′ CAAGCAGAAGACGGCATACGAGAT
Complementary to P7
Primer2:
(SEQ ID NO: 9)
5′ AATGATACGGCGACCACCGAGATCTACACnnnnnnnnACACTCT
TTCCCTACACGAC

P5—Partial Read 1

i5 Barcode

Ex Vivo Differentiation of BMDCs

Ex vivo grown BMDCs, bone marrow cells were obtained by plating total bone marrow cells at a density of 200,000 cells/ml on non-tissue culture treated plastic dishes (10 ml medium per plate). At day 2, cells were fed with another 10 ml medium per dish. At day 5, cells were harvested from 15 ml of the supernatant by spinning at 1400 rpm for 5 minutes; pellets were resuspended with 5 ml medium and added back to the original dish. Cells were fed with another 5 ml medium at day 7. BMDC medium contains: RPMI (Gibco) supplemented with 10% heat inactivated FBS (Gibco), β-mercaptoethanol (50 uM, Gibco), L-glutamine (2 mM, Biological Industries) penicillin/streptomycin (100 U/ml, Biological Industries), MEM non-essential amino acids (1×, Biological Industries), HEPES (10 mM, Biological Industries), sodium pyruvate (1 mM, Biological Industries), and GM-CSF (20 ng/ml; Peprotech).

Mouse ESC Tissue Culture and Handling

Mouse B6×BCA F1 ESC line (carrying ΔPE Oct4-GFP transgenic reporter—Addgene 52382) was expanded in feeder free 0.2% Gelatin (SigmaAldrich) coated plates in one of the following two naïve pluripotency conditions:

1. Serum/LIF conditions (also known as—mouse metastable naïve conditions): 425 ml High-Glucose DMEM (Invitrogen—41965), 15% USDA certified and heat inactivated Fetal Bovine Serum (FBS) (Biological Industries), 1 mM L-glutamine (Biological Industries), 1% nonessential amino acids (Biological Industries), 0.1 mM b-Mercaptoethanol (Invitrogen), penicillin/streptomycin (Biological Industries), Sodium Pyruvate (Biological Industries) and 20 ng/ml recombinant human LIF (produced in-house).

2. 2i/LIF conditions (also known as—mouse naïve ground state conditions): 240 mL DMEM/F12 (Invitrogen 21331), 240 mL Neurobasal (Invitrogen—21103), 5 mL N2 supplement (Invitrogen—17502048), 5 mL B27 supplement (Invitrogen 17504-044), 1 mM glutamine (Biological Industries), 0.1 mM non-essential amino acids (Biological Industries), 0.1 mM (3-mercaptoethanol (Sigma), penicillin-streptomycin (Biological Industries), 50 μg/mL BSA (GIBCO Fraction V—15260-037), 20 ng/ml recombinant human LIF and 2 small-molecule inhibitors (2i): CHIR99021 (GSK3i—3 μM—Axon Medchem 1386) and PD0325901 (MEKi—1 μM—Axon Medchem 1408).

Cells were expanded in 20% 02; 5% CO2 at 37° C. Cells were passage following single cells trypsinization (0.25% Trypsin—Biological Industries) every 4-5 days. Exclusion of Mycoplasma contamination was monitored and conducted by monthly routine tests with Mycoalert kit (LONZA).

Adult Tissues

Brain, liver, kidney and lungs from euthanized C57BL/6J female mice (8 to 12 weeks old) were extracted and washed 3 times with 10 ml of ice cold PBS-Pi. Organs were then crosslinked in 10 ml of PBS/1% formaldehyde. Crosslinking was stopped by adding glycine to 125 mM and incubating 5 min. Media was discarded and organs were washed 3× with ice-cold PBS-Pi, snap-frozen and stored at −80° C.

Whole tissues were thawed in 10 ml of LB1-Pi buffer and cut in small pieces using scissors. Organs were incubated at 4° C. in LB1-Pi buffer for 10 min, pelleted by centrifugation at 12000×G/4° C. for 10 min and re-suspended in 1 ml of RIPA-I buffer. Organs were then sonicated using similar parameters than for BMDCs or ES cells. 50 μl of each organ extract were decrosslinked, SPRI bead purified and the DNA concentration was measured. We used for each primary IP extract amounts equivalent to 2 μg of DNA.

Processing of Co-ChIP Data

All Co-ChIP libraries were sequenced using the IlluminaNextSeq 500. Reads were aligned to the mouse reference genome (mm9, NCBI 37) using Bowtie2 aligner version 2.2.5 with default parameters. The Picard tool MarkDuplicates from the Broad Institute (broadinstitutedotgithubdotio/picard/) was used to remove PCR duplicates. For scatterplots and model analysis, raw reads were counted in a sliding window across the entire genome of 1 kb with 500 bp overlap. To identify regions of enrichment (peaks) from Co-ChIP reads of each PTM pair, we used the HOMER package makeTagDirectory followed by the findPeaks command with the histone parameter (PMID: 20513432). Normalized profiles were generated using makeBigWig.pl script from the HOMER package and visualized using the WashU EpiGenome Browser.

Estimating Co-Occurrence from Read Counts

Since all 14 PTMs antibodies were pooled together after the first barcoding step and then split into 6 equal aliquots for a second IP step, it can be assumed that each secondary IP was performed on a similar input with the same distribution of PTM¹ barcodes. To estimate the co-occurrence for 14×5 PTMs directly from the relative abundance of read counts, the present inventors first counted reads from the H3 pool (H3 as a secondary antibody) for each PTM¹, assuming this represented the background distribution of PTM¹ fragments in the pooled chromatin. They then compared the proportion of read counts from each PTM¹ in a given PTM² pool to the proportion in the H3 pool. The ratio of these numbers is an estimation of the co-occurrence of each PTM¹-PTM² pair. Since each pool received different total read count there is an implicit scaling factor.

Clustering Analysis

For the clustering of Co-ChIP read counts in FIG. 2B, the present inventors the matlab K-means algorithm with the correlation distance metric. K was chosen at 15 because lower values failed to identify all meaningful clusters and higher values subdivided existing clusters. They selected 37 pairs based on their level of co-occurrence. They removed regions from the clustering that had fewer than 15 reads for any one PTM pair, leaving a total of 158,200 regions (1 kb each). RNA expression data was taken from Garber et al.²⁴, and regions were associated to the nearest gene within 50 kb to produce the box and whisker plot for each cluster. For discover of super-enhancers, the original strategy as presented in Whyte et al. was used²¹. ChIP-seq data for Med12, PU.1 and Cebpb from²⁴ (master transcription factors and mediator) were used as input to the HOMER findPeaks program with the ‘-style super’ option.

Multiplicative Model to Estimate Co-Occurrence from Conventional ChIP-Seq Pairs

In order to estimate the expected distribution of co-occurrence of each PTM pair, a simple multiplicative behavior (independent observations) was used. For the PTM pair, PTM¹-PTM² X¹=x_{i=1 . . . N}¹, X²=x_{i=1 . . . N}² was defined as the read counts for PTM¹ and PTM² from conventional ChIP where N is the number of 1 kb sliding windows and Y^{1,2} is the Co-ChIP read count for the pair. The predicted Co-ChIP counts are defined as:

ŷᵢ^{1,2}=f(x,β)=β₁*(xᵢ¹)^{β}^{₂}*(xᵢ²)^{β}^{₃}

The model parameters were estimated to minimize the least square equation, using nonlinear regression (nlinfit in matlab):

$${\sum }_{i = 1}^{N} {( {y}_{i} - f ( x , β ) )}^{2}$$

To choose the threshold for exclusion/inclusion the log fold change (FC) between model and measurements was first calculated after adding a constant of 10 reads per region to avoid significant FC in low coverage regions. Next, the fold-difference were transformed into Z-scores, and regions with Z-score above 2 or below −2 as exclusion/inclusion were classified. Several constants and Z-score cutoff were tested and essentially the same enrichment results were obtained.

To further validate the global trends of exclusion/inclusion with respect to genomic elements (promoters/enhancers), a non-parametric method (k-nearest neighbors with Gaussian kernel) was used to calculate the average co-occurrence signal as a function of the two single ChIP measurements. K=50, 100, 200 was tested and very similar results were obtained.

Bivalent Data Analysis of 4 Adult Tissues and ES Cells

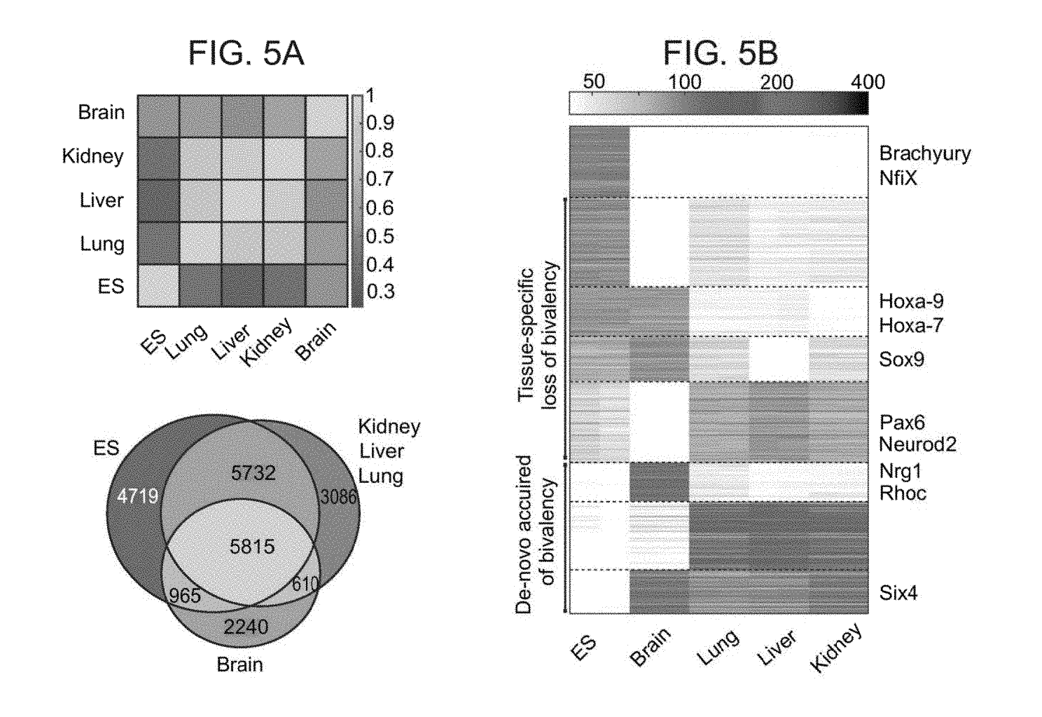

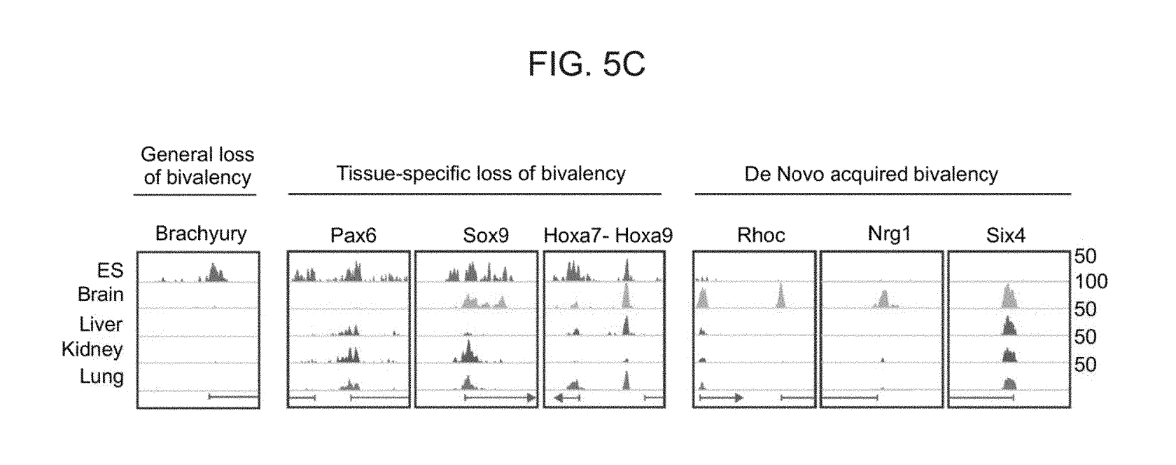

Co-ChIP of 4 adult tissues and ES cells was processed as described above (Processing of Co-ChIP data). The present inventors limited their bivalent analysis to high confidence regions where the read count of both replicates were within the top 25th percentile. Union peaks file were generated by combining and merging overlapping peaks in all tissues. Clustering was performed using matlab K-means algorithm. For the Venn diagrams in FIG. 5A, regions were classified as shared if the intensity of the region in one tissue was <2-fold higher than the intensity of the region in the second tissue. Similarly, a region shared between set of tissues was considered if the difference between the maximum and minimum intensities in these populations is <2-fold.

Results

Co-ChIP: A Genome Wide Method for Combinatorial Pairwise ChIP-Seq

To explore the genome-wide co-occurrence of pairs of histone marks, the present inventors developed a method for combinatorial pairwise chromatin immunoprecipitation (Co-ChIP). The basis of this technology is the coupling of the immunoprecipitation and direct histone barcoding¹⁷. During Co-ChIP, the chromatin is immobilized on magnetic beads coupled to the relevant set of post-translational modification (PTM) antibodies, and the chromatin fragments are indexed with DNA adaptors (FIG. 1A). Once the chromatin fragments have been barcoded, the first antibody is inactivated and released from the chromatin: this approach enables pooling of nucleosomes indexed for different primary PTMs. The pool of barcoded chromatin can then be split into an array of secondary PTM antibodies for a second immunoprecipitation step that enriches for nucleosomes with co-occurrence of both modifications (FIG. 1A). As a reference, the antibody for the H3 histone can be added in this step instead of a second PTM to act as a control for normalization. For sequencing, Co-ChIPed DNA is amplified by PCR and barcoded with an additional index representing the second PTM enabling each pair of histone PTMs to be identified with a unique combination of indexes.

To test whether Co-ChIP can be used for measuring co-occurrence of pairs of histone marks, the present inventors first applied it on BMDC to detect well known associations between histone PTMs within defined genomic regions: H3K4me3-H3K27ac for active promoters and H3K4me3-H3K27me3 for poised (bivalent) promoters. Mapping the indexed sequencing reads, they identified with high resolution and sensitivity the presence of both H3K27ac and H3K4me3 at 34,167 regions including known active BMDC promoters, such as in the tumor necrosis factor alpha (TNF) locus. They also noted the presence of the H3K27me3-H3K4me3 pair at BMDC poised/repressed genes such as the key B cell developmental gene Ebf1 and the Hoxa cluster (FIG. 1B). Generally, the raw data from Co-ChIP directly separates active promoters from poised promoters without the need to computationally integrate several experiments as each region is exclusively marked by one Co-ChIP combination. To limit nonspecific binding, validated ChIP antibodies that have been screened to minimize false positive signal from other histone modifications were used¹⁸. In order to estimate the noise of the present method, Co-ChIP was performed on a pair of mutually exclusive marks, H3K27ac and H3K27me3, and it was observed that the reads mapped randomly across the genome representing a background distribution without significant peaks (FIG. 1B). These results rule out the possibility of antibody carryover or nonspecific DNA binding from the first IP into the second IP as a major source of contamination in the process.

Next the sensitivity of Co-ChIP to the order in which the PTM immunoprecipitations are performed was tested. The Co-ChIP profile of the pair H3K4me3^{1Ab}-H3K27ac^{2Ab} was compared by switching the order to H3K27ac^{1Ab}-H3K4me3^{2Ab}. A very high correlation (r=0.9735) between both of these marks was observed as well as between other PTM pairs such as H3K4me3-H3K27me3, H3K4me2-H3K18ac, indicating that the order of antibodies used for Co-ChIP does not significantly impact the identification of co-occurring regions for most PTM pairs (FIG. 1C). To further test the reproducibility of Co-ChIP, the profiles obtained for the pair H3K27ac^{1Ab}-H3K4me3^{2Ab} were compared using three different commercial antibodies for the H3K4me3 epitope and a high correlation between the three different clones was confirmed (all pairwise correlations >0.97; FIG. 1D). Finally, the present inventors evaluated whether Co-ChIP can be used to probe histone mark and transcription factor interactions. To test this scenario, the present inventors used Co-ChIP for three sequence-specific factors (Pu.1, CTCF and Cebpb) with three histone marks (H3K4me2, H3K4me3 and H3K27ac) and identified specific peaks for each TF-modification pair with low background signal (FIG. 1E). We find that Pu.1 and CTCF preferentially bind adjacent to enhancers, as evidenced by overlap with H3K4me2- and H3K27ac-modified nucleosomes, whereas Cebpb preferentially binds next to promoters (H3K4me3-modified nucleosomes)^{19,20} (FIG. 1F). Together, these results show that Co-ChIP is a sensitive and specific method to characterize genome-wide co-occurrence of histone PTMs and transcription factors.

Measurement of the Co-Occurrence of 70 Histone Pairs Identify Novel Interactions

The present inventors applied Co-ChIP to evaluate the global co-occurrence of 14 primary histone marks, representing the major acetylation and methylation events, against 5 secondary histone marks (H3K4me1/me2/me3, H3K27ac and H3K18ac) in BMDCs. The antibody for the H3 histone was used as a reference control for normalization of the Co-ChIP in order to account for differences in IP yield of each primary PTM, due to varying antibody affinities and differing relative abundances of the histone marks across the genome. To quantify the relative abundance of each PTM pair, the present inventors calculated the ratio of read counts from the Co-ChIP (PTM¹-PTM²) compared to the read counts from the H3 control (PTM¹-H3). Comparing the relative abundances to the pairwise correlations of conventional single PTM ChIP revealed an overall agreement between these two measurements (FIG. 2A). For example, the relative abundance for all 14 PTMs with H3K27ac as the secondary antibody highly correlates with the pairwise correlations of conventional ChIP. However, further analysis of the co-occurrence of several histone modification pairs show disagreement between Co-ChIP measurements and the results obtained from conventional ChIP signals as discussed below.

Clustering of the histone modification pairs revealed 15 distinct clusters corresponding to specific chromatin states which vary by genomic position and the expression profiles of neighboring genes (FIG. 2B, Methods). Plotting conventional single ChIP signal for each modification over the 15 clusters, cluster specific enrichment for several combinations was observed that cannot be predicted from overlaying the respective conventional ChIP signals, some examples include the co-occurrence of H3K9me1-H3K4me1 in cluster IV, H3K36me2-H3K4me1 in cluster 5 or the dynamic levels of acetylation in H3K4me2 and H3K4me3 regions observed across clusters 1-4. Cluster 1, which is enriched for regions that are distal to the TSS, exhibited strong co-occurrence of several acetylation marks with H3K4me1/me2 as well as co-occurrence of multiple acetylation marks. This cluster is associated with high expression of neighboring genes and further analysis indicates that it is enriched for “super-enhancer” regions containing key BMDC regulatory genes, such as Pu.1 and Junb²¹ (FIG. 2B). Cluster 2 shows similar patterns with lower levels of the signature PTMs. The co-occurrence of H3K9me1-H3K27ac was most prominent in these two clusters, and may represent a specific association of this pair in these locus control regions. On the other hand, co-occurrence between H3K4me1 and H3K9me1 was more even across distal elements (Clusters 1-5) regardless of their activity. Cluster 6 and 7 were enriched for repressed genes, for example Ebf1, and are associated with poised enhancers and promoters, respectively, that display co-occurrence of H3K27me3 with H3K4me1, H3K4me2 and H3K4me3. Cluster 8 was enriched with active gene bodies and elongation marks such as H3K36me2/me3 together with H3K4me2 and H3K27ac. Globally, the present inventors did not observe co-occurrence in their analysis of histone marks that are associated with non-overlapping genomic locations in single ChIP; for example Co-ChIP of H3K4me3, a mark which is enriched in promoters regions, and H3K36me3, which is mainly localized to the transcribed gene body, display very little overlap as would be expected from modifications affiliated with mutually exclusive regions. On the other hand, interacting modifications such as H3K27ac and H3K18ac display overlapping Co-ChIP profiles that are similar to the profile of each mark separately.

Probing for Enrichment or Depletion in the Interactions Between Pairs of Histone Marks

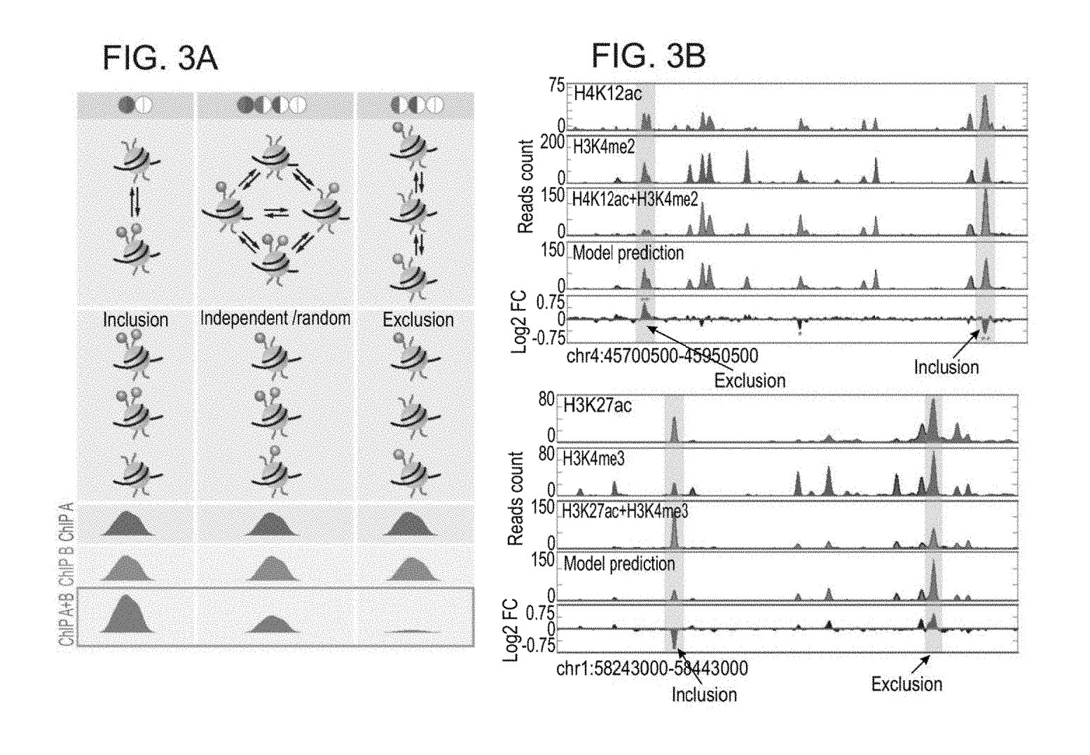

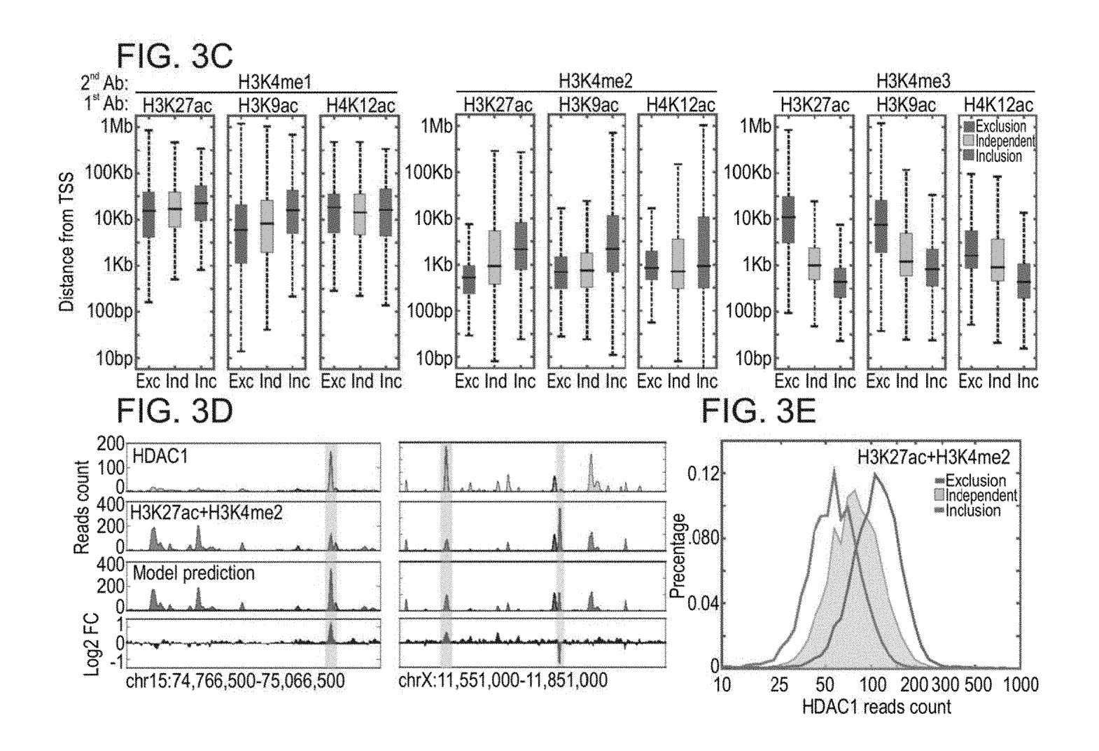

It was hypothesized that some combinations of histone marks will share the same genomic positioning, but tend not to co-occur due to cell-to-cell heterogeneity. It was reasoned that the genomic positions in which discrepancies between conventional ChIP and Co-ChIP were identified in a homogenous cell population may provide insights into the mechanism by which histone marks are deposited and removed. If these processes of removal and depositing of marks are independent, it can be expected that the Co-ChIP signal would resemble the random overlap of the individual marks (multiplication of their frequencies). If there is exclusion between two histone marks (i.e. deposition of one checks the other), the Co-ChIP signal should be lower than the random overlap; however if marks behave in an “inclusive” way (i.e. deposited and removed together) a higher Co-ChIP signal is expected compared to the random overlap (FIG. 3A). Moreover, the relationship between marks may vary depending on the identity of the genomic region. To quantify the interactions between histone marks across the genome, an analytical model was designed to predict the co-occurrence of histone marks based on the conventional single ChIP signals. A simple multiplicative model with only 3 scaling parameters was assumed and the present inventors searched for discrepancies between Co-ChIP data and the model prediction (Methods). The free parameters of the model (describing exponential scaling factors for IP efficiency) were estimated to maximize the likelihood of the experimental observations.

The present inventors applied their model on histone pairs that occupy common regions but also have unique regions that are not shared. They excluded all pairs that share a large fraction of regions (such as H3K27ac and H3K18ac) or are mutually exclusive (such as H3K4me3 and H3K36me3), since in such cases the model will not provide meaningful insights (Methods). In general, they found good agreement between the predictions and the Co-ChIP signal (correlation coefficient >0.9). They classified peaks that show higher or lower Co-ChIP signal than the predicted value as inclusion or exclusion of a specific pair of marks (FIG. 3B). They next searched for genomic features characterizing regions showing exclusion versus inclusion of histone modification co-occurrence. Examining various acetylations versus H3K4 methylation revealed that regions showing exclusion of acetylation with H3K4me1/2 were closer on average to the TSS than inclusion regions: this suggests that inclusion between mono/di H3K4 methylation and acetylation was more common at enhancers, while exclusion was more common at promoters. H3K4me3 co-occurrence with acetylation displayed the opposite pattern: inclusion tended to be closer to the TSS (FIG. 3C). To further validate that their results were not skewed by non-linear antibody specific effects or saturation effects, they used an alternative, non-parametric method (k-nearest neighbors) to calculate the expected co-occurrence signal as a function of the two single ChIP measurements. They detected the same trends of exclusion/inclusion that were found using the parametric models. The present inventors hypothesize that these findings may be a result of either temporal dynamics and/or cell-to-cell variability. Mechanistically, differences in the activity of chromatin modifiers at different genomic elements and the specific chromatin state at which they operate may alter the co-occurrence of marks in a region-specific manner.

To further disentangle the relationship between acetylation and H3K4 methylation, the present inventors profiled the binding of a major chromatin modifier, histone deacetylase 1 (HDAC1), in BMDC using ChIP-seq and compared the binding profile of HDAC1 to the inclusion/exclusion patterns of acetylation and H3K4 methylation (FIG. 3D). Interestingly, it was found that regions enriched with HDAC1 tend to display exclusive behavior while regions that are HDAC1-depleted are more inclusive—i.e. that the action of HDAC1 tends to preclude the methylation of these regions (FIG. 3E). Similarly, the dynamics of specific chromatin modifiers may explain the co-occurrence interactions of other histone marks at specific genomic loci. Although such behaviors could not be studied by conventional ChIP-seq experiments, Co-ChIP can discriminate between transient and stable patterns of co-occurrence. These dynamics may play important roles in gene expression and epigenetic regulation.

Genome-Wide Characterization of Bivalent Domains in ES Differentiation

Pioneering work on ES cells identified regions with overlap of H3K4me3 (activation) and H3K27me3 (repression) marks on promoters of key developmental genes¹¹. Later work has validated that these are genuine co-occurrence of the marks on a handful of regions using reChIP²²; however, to date there are no genome-wide studies profiling the bivalent state.

Using Co-ChIP, the present inventors set out to characterize bivalent domains and their dynamics during development. Previous studies used conventional H3K4me3 and H3K27me3 ChIP-seq to show induction of bivalency when naive ES cells (Cultured with 2 inhibitors for MEK and GSK3; 2i/LIF) are primed for development towards the different lineages via transfer into Serum/LIF conditions (ES Serum/LIF). The present inventors used Co-ChIP to profile genome-wide bivalent domains of ES cells in 2i/LIF vs. Serum/LIF conditions. As a comparison, they also profiled the dynamics of co-occurrence of H3K27ac and H3K4me3, which in theory should be mutually exclusive to the classical bivalent domains. They found that, in both cell states, critical pluripotent genes, such as Nanog and Sall4, are in an active state (high H3K27ac-H3K4me3) and show no bivalent signal (H3K27me3-H3K4me3) (FIGS. 4A-B). The only exception is Oct4 which is both actively marked and bivalent in 2i/LIF and Serum/LIF conditions, potentially due to heterogeneity in the promoter of Oct4 alleles (active or poised) in individual cells from the population. Indeed, single cells studies of ES cells identify variability in Oct4 gene expression²³. Analysis of important developmental genes such as Gata3, Gata4, Tal1 and the Hox clusters showed the expected induction of bivalency in the more primed ES cell population with minor signal in the 2i/LIF naive ES cell population (FIG. 4A). Interestingly the present inventors noticed that high bivalent signal is not restricted to the promoter but is also observed inside the gene bodies or several kilobases upstream of the TSS. Scatterplots of the H3K27ac-H3K4me3 signal in both populations detects mostly regions with comparable signal in 2i/LIF and Serum/LIF ES cells (including Sa114) with few phase-specific regions. In contrast, the H3K27me3-H3K4me3 signal is primarily seen in Serum/LIF conditions that induce a bivalent signature in 12.9% of the H3K4me3 regions (FIG. 4B). Gene ontology analysis of genes associated with the bivalent marks identifies enrichment for developmental functions and transcription factors. Together, the present analyses uncover massive induction of bivalency in the transition from the naïve toward a relatively more primed state of ES cells, with the exception of Oct4 and Sfi1 that are bivalent in both.

Bivalency is Gained and Lost in Adult Tissues

Since Co-ChIP measures co-occurrence of marks on the same DNA molecule, alleviating biases associated with cell heterogeneity, it allows the user to study the bivalency status of heterogeneous tissues. In this context, Co-ChIP is a major advancement since the study of bivalency in tissues cannot be approximated using computational methods which cannot distinguish between overlaps in time and space. To explore the bivalency present in the adult tissues, 4 mouse tissues were selected, brain, liver, lung and kidney, and the genome-wide co-occurrence of H3K27me3-H3K4me3 on the same nucleosome was measured. These experiments generated a catalog of bivalent regions, which together with the ES bivalent domains make a total of 23,167 bivalent regions with 2240 unique brain regions and 3086 unique kidney, liver and lung regions (FIG. 5A). Using their catalog of bivalent regions, the present inventors first analyzed if bivalent patterns are linked to the developmental origins. To test this hypothesis, they performed pairwise correlation of the bivalent profiles between the four tissues and ES cells. Coherent with the developmental origin, they observe that kidney, liver and lung show very similar bivalent status as compared to brain and ES cells (FIG. 5A).

To systematically evaluate changes in bivalent regions across ES cells and tissues, all bivalent regions were clustered together. A global decrease of bivalency in the adult tissues was observed as compared to the primed ES cells. Of the 17,231 bivalent domains characterized in primed ES cells, 66% are lost in at least one of the four tissues (FIGS. 5A-B). The Brachyury (also known as T) gene, which codes for a transcription factor with critical roles during early development is one such example: it is bivalent in ES cells but not in any of the tissues (FIG. 5C). A subset of the lost bivalent regions (38%) are selectively lost in a particular tissue but maintained in all others (FIGS. 5A-B). Interestingly, the present inventors found in this group known tissue-lineage factors including Pax6 for brain, FoxAl for liver and Hoxa7 for kidney (FIG. 5C). In these cases, the bivalent status of the locus is lost only in the relevant tissue where the gene is expressed and functionally important, and maintained in the rest of the tissues where the transcription factor is not expressed.

In addition to loss of bivalency, the present analysis also identifies bivalent regions that are acquired de novo in adult tissues (FIGS. 5B-C). The emergence of bivalency after ES priming is not negligible, 5936 newly acquired bivalent regions from the more primed ES state (Serum/LIF conditions) were detected, which represents 25% of all bivalent regions detected in our study. Two major clusters of de novo bivalent regions were found: one consisted of brain-specific regions and the other was shared among liver, kidney and lung. These genes are generally expressed in the relevant tissue in a tissue-specific manner (FIGS. 5B and 5C), which may suggest cell-type-specific regulation within the tissue. A group of de novo bivalent regions shared by all tissues was also detected (FIG. 5C). Analysis of this group identified bivalent domains present at genes such as Six4, which is a homeobox transcription factor involved in many developmental processes (olfactory, muscle regeneration, kidney, etc). Such pleiotropic regulatory factors must be tightly regulated and bivalency may be a potential mechanism to achieve this goal. Together, the present characterization of adult bivalency revealed two important insights; i) the loss of co-occurrence of H3K4me3-H3K27me3 regions in a tissue-specific manner during development, and ii) the emergence of de novo bivalent regions in adult tissues to prime various tissue-specific genes.

### DISCUSSION

Chromatin serves as a nexus connecting the genome with different environmental inputs. Characterizing the regulatory elements and the epigenetic features associated with them is critical for understanding genome function and regulation. An important aspect of chromatin regulation is the interactions between different histone PTMs, their dynamic behavior and regulation. To probe this important question, previous approaches either assumed homogeneity in the population or empirically measured such interactions at a few particular loci. Despite these important efforts, a robust technology to measure histone PTM interactions in a global and quantitative manner is still lacking. A new method is described herein, Co-ChIP that enables genome-wide, reproducible and sensitive measurements of the co-occurrence of different histone marks and transcription factors on the same molecule. Co-ChIP was successfully applied to both cell cultures and whole organs and it was shown to be broadly applicable across organisms and tissues.

It was found that different antibody efficiencies in chromatin pull-down will impact the quality of the results. Nevertheless, it was found that with mindful design, starting with the more efficient antibody, this can be controlled. For example when probing for co-occurrence of a chromatin modification and a TF, it is important to start with the chromatin modification and use the TF as a secondary antibody to enable proper complexity and efficient barcoding of the chromatin.

In most cases, when profiling the epigenetic landscape such as in tissues or cancer samples, a homogenous populations of cells is not expected and hence the observation of overlapping chromatin marks may be the result of population heterogeneity. The present application of Co-ChIP to characterize the dynamics of bivalent domains at different developmental stages identifies massive induction of bivalency in primed ES cells. A large portion of these bivalent regions is lost in a tissue-specific manner, but the emergence of new bivalent domains in various mature tissues, especially in the brain was also identified. In general, these de novo bivalent domains are associated with genes that are differentially expressed in the tissue, suggesting bivalency is a means of gene priming in adult tissues. These results demonstrate the potential of Co-ChIP to identify important insight on developmental and stem cell biology.

Through profiling of 70 pairwise interactions of histones in BMDC, the present inventors identified previously unknown co-occurrences of many histone modifications. Among these pairs, the co-occurrence of H3K4me1/me2 with H3 acetylation at active enhancers, H3K4me1 and H3K27me3 at repressed/poised enhancers and many others were discerned. In addition, co-occurrence of chromatin marks associated with particular genomic elements including the co-occurrence of H3K9me1 with H3K27ac at super-enhancers was uncovered. The present inventors then showed that Co-ChIP data can uncover dynamic relationships between different chromatin modifications. In conventional ChIP, H3K4me2/me1 and H3K27ac appear in the same genomic regions. In contrast, the co-occurrence of these two marks as measured by Co-ChIP show inclusive interactions in enhancers versus promoters. Interestingly, it was found that in regions distal to the TSS, acetylated nucleosomes stably interact with H3K4me1/2, whereas their interactions with H3K4me3 are more transient and seem to have a higher turnover. This is in line with recent reports identifying Rack? and Kdm5C as important regulators of distal H3K4me3 signals¹⁹. Taken together, Co-ChIP has been demonstrated to be a powerful tool to probe the previously hidden dynamics and interactions between different chromatin modifications, paving the way for improved understanding of the histone code and its relevance in stem cell biology, development and disease.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

### REFERENCES

1. Turner, B. M. Cellular memory and the histone code. Cell 111, 285-291 (2002).
2. Kouzarides, T. Chromatin modifications and their function. Cell (2007).
3. Creyghton, M. P. et al. Histone H3K27ac separates active from poised enhancers and predicts developmental state. Proc. Natl. Acad. Sci. U.S.A. 107, 21931-21936 (2010).
4. Shlyueva, D., Stampfel, G. & Stark, A. Transcriptional enhancers: from properties to genome-wide predictions. Nat. Rev. Genet. 15, 272-286 (2014).
5. Jenuwein, T. & Allis, C. D. Translating the histone code. Science (2001).
6. Strahl, B. The language of covalent histone modifications. Nature (2000).
7. Dion, M. F., Altschuler, S. J. & Wu, L. F. Genomic characterization reveals a simple histone H4 acetylation code. in (2005).
8. Schreiber, S. L. & Bernstein, B. E. Signaling network model of chromatin. Cell (2002).
9. Yan, L. et al. Epigenomic landscape of human fetal brain, heart, and liver. Journal of Biological . . . (2015).
10. Kundaje, A., Meuleman, W., Ernst, J., Bilenky, M. & Yen, A. Integrative analysis of 111 reference human epigenomes. Nature (2015).
11. Bernstein, B. E. et al. A bivalent chromatin structure marks key developmental genes in embryonic stem cells. Cell 125, 315-326 (2006).
12. Grandy, R. A., Whitfield, T. W. & Wu, H. Genome-wide Studies Reveal that H3K4me3 Modification in Bivalent Genes is Dynamically Regulated During the Pluripotent Cell Cycle and Stabilized Upon . . . and cellular biology (2015).
13. Ernst, J. et al. Mapping and analysis of chromatin state dynamics in nine human cell types. Nature 473, 43-49 (2011).
14. Ernst, J. & Kellis, M. ChromHMM: automating chromatin-state discovery and characterization. Nat. Methods (2012).
15. Guan, X., Rastogi, N., Parthun, M. R. & Freitas, M. A. Discovery of Histone Modification Crosstalk Networks by SILAC Mass Spectrometry. Mol. Cell Proteomics (2013). doi: 10.1074/mcp.M112.026716
16. Britton, L., Gonzales-Cope, M. & Zee, B. M. Breaking the histone code with quantitative mass spectrometry. Expert review of . . . (2011).
17. Lara-Astiaso, D. et al. Chromatin state dynamics during blood formation. Science (2014). doi:10.1126/science.1256271
18. Egelhofer, T. A., Minoda, A., Klugman, S. & Lee, K. An assessment of histone-modification antibody quality. Nature structural & . . . (2011).
19. Ghisletti, S. et al. Identification and characterization of enhancers controlling the inflammatory gene expression program in macrophages. Immunity 32, 317-328 (2010).
20. Heinz, S. et al. Simple combinations of lineage-determining transcription factors prime cis-regulatory elements required for macrophage and B cell identities. Mol Cell 38, 576-589 (2010).
21. Whyte, W. A., Orlando, D. A., Hnisz, D., Abraham, B. J. & Lin, C. Y. Master transcription factors and mediator establish super-enhancers at key cell identity genes. Cell (2013).
22. Furlan-Magaril, M. & Rincón-Arano, H. Sequential chromatin immunoprecipitation protocol: ChIP-reChIP. DNA-Protein Interactions: . . . (2009).
23. Kumar, R. M. et al. Deconstructing transcriptional heterogeneity in pluripotent stem cells. Nature 516, 56-61 (2014).
24. Garber, M. et al. A high-throughput chromatin immunoprecipitation approach reveals principles of dynamic gene regulation in mammals. Mol Cell 47, 810-822 (2012).

1. A method of analyzing DNA molecules in a cell sample, said DNA molecules having DNA binding moiety signatures which are defined by at least two non-identical DNA binding moieties, the method comprising:
(a) labeling DNA molecules of the cell sample with a label that indexes the identity of at least two of the DNA binding moieties of said signatures so as to generate subpopulations of differentially labeled DNA molecules, each subpopulation being in a separate container, wherein said labeling indexes one DNA binding moiety per DNA molecule;
(b) labeling said differentially labeled molecules with another label which indexes the identity of another DNA binding moiety of said signature; and
(c) analyzing the DNA comprising said first label and said second label.

2. The method of claim 1, wherein said label is a nucleic acid label.

3. The method of claim 1, wherein said labeling of step (a) comprises attaching no more than one label per DNA molecule.

4. The method of claim 1, wherein said labeling comprises end-labeling.

5. The method of claim 1, wherein said labeling of step (b) comprises attaching no more than one label per DNA molecule.

6. The method of claim 1, further comprising repeating step (b) using an additional label prior to step (c).

7. The method of claim 1, wherein neither said first DNA binding moiety nor said second DNA binding moiety bind to more than 50% of the DNA of the sample.

8. The method of claim 1, further comprising pooling said subpopulations to generate a pooled sample of differentially labeled DNA molecules following step (a) and prior to step (b).

9. The method of claim 1, further comprising shearing the DNA of the cell sample prior to step (a).

10. The method of claim 1, wherein said analyzing comprises sequencing said DNA.

11. The method of claim 1, further comprising analyzing said DNA binding moieties following step (b).

12. The method of claim 1, wherein said DNA is no longer than 500 bases.

13. The method of claim 1, wherein said DNA binding moiety is a DNA binding protein.

14. The method of claim 13, wherein said DNA binding protein is a histone.

15. The method of claim 13, wherein said DNA binding protein is a transcription factor.

16. The method of claim 1, wherein said DNA binding moiety is a drug.

17. The method of claim 1, wherein said sample is derived from cells of a single type or line.

18. The method of claim 14, wherein said histone is a post-translationally modified histone.

19. The method of claim 18, wherein said post-translationally modified histone is a methylation or acetylation.

20. The method of claim 19, wherein said post-translationally modified histone is selected from the group consisting of H3K4me1, H3K4me2, H3K4me3 and H3K27ac.

21. A kit for immunoprecipitating a DNA-protein complex comprising:
(i) at least one antibody which specifically binds to a transcription factor;
(ii) at least one antibody which specifically binds to a post-translationally modified histone; and
(iii) a DNA labeling agent.

22. The kit of claim 21, further comprising an antibody for immobilizing at least 50% of the chromatin of a cell.

23. The kit of claim 21, further comprising at least one agent selected from the group consisting of an RNA polymerase, a DNAse and a reverse transcriptase.

24. The kit of claim 21, further comprising a plurality of barcode DNA sequences.

25. The kit of claim 21, further comprising a solid support for immobilizing said at least one antibody.

26. The kit of claim 21, further comprising at least one component selected from the group consisting of a crosslinker, a protease enzyme and a ligase.


SEQUENCE LISTING (10 sequences)

SEQ ID NO: 1 - DNA, 33 bp, Artificial Sequence
  Universal ChIP adaptor NA sequence
  misc_feature (32)..(33): phosphorothioate bond
acactctttccctacacgacgctcttccgatct

SEQ ID NO: 2 - DNA, 65 bp, Artificial Sequence
  Indexed adaptors with 5′ phosphorylation
  misc_feature (1)..(1): 5′  phosphorylated
gatcggaagagcacacgtctgaactccagtcacctaccaggatctcgtatgccgtcttctgcttg

SEQ ID NO: 3 - DNA, 65 bp, Artificial Sequence
  Indexed adaptor with 5′ phosphorylation
  misc_feature (1)..(1): 5′ phosphorylation
gatcggaagagcacacgtctgaactccagtcaccatgcttaatctcgtatgccgtcttctgcttg

SEQ ID NO: 4 - DNA, 65 bp, Artificial Sequence
  Indexed adaptor with 5′ phosphorylation
  misc_feature (1)..(1): 5′ phosphorylation
gatcggaagagcacacgtctgaactccagtcacgcacatctatctcgtatgccgtcttctgcttg

SEQ ID NO: 5 - DNA, 65 bp, Artificial Sequence
  Indexed adaptor with 5′ phosphorylation
  misc_feature (1)..(1): 5′ phosphorylation
gatcggaagagcacacgtctgaactccagtcactgctcgacatctcgtatgccgtcttctgcttg

SEQ ID NO: 6 - DNA, 33 bp, Artificial Sequence; identical to SEQ ID NO: 1
  Single strand DNA oligonucleotide

SEQ ID NO: 7 - DNA, 66 bp, Artificial Sequence
  Single strand DNA oligonucleotide
  misc_feature (35)..(42): n is a, c, g, or t
agatcggaagagcacacgtctgaactccagtcacnnnnnnnnatctcgtatgccgtcttctgcttg

SEQ ID NO: 8 - DNA, 24 bp, Artificial Sequence
  Single strand DNA oligonucleotide
caagcagaagacggcatacgagat

SEQ ID NO: 9 - DNA, 56 bp, Artificial Sequence
  Single strand DNA oligonucleotide
  misc_feature (29)..(36): n is a, c, g, or t
atgatacggcgaccaccgagatctacacnnnnnnnnacactctttccctacacgac

SEQ ID NO: 10 - DNA, 35 bp, Artificial Sequence
  T7 RNA polymerase promoter sequence
cgattgaggccggtaatacgactcactataggggc